(19) United States
(12) Patent Application Publication
(10) Pub. No.: US 2005/0214223 A1
(43) Pub. Date: Sep. 29, 2005

(54) Abuse-safeguarded dosage form

(75) Inventors: Johannes Bartholomaeus, Aachen (DE); Klaus-Dieter Langner, Aachen (DE); Heinrich Kugelmann, Aachen (DE)

Correspondence Address: CROWELL & MORING LLP;INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 11/113,118

(22) Filed: Apr. 25, 2005

Related U.S. Application Data
(63) Continuation of application No. PCT/EP03/11784, filed on Oct. 24, 2003.

(30) Foreign Application Priority Data
Oct. 25, 2002 (DE) DE 102 50 084.3

(51) Int. Cl. A61K 49/00; A61K 31/515; A61K 31/454

(52) U.S. Cl. 424/10.1; 514/270; 514/326

(57) ABSTRACT

A pharmaceutical dosage form that is safeguarded against abuse containing at least one active substance that is susceptible to abuse and at least two of the following constituents (a) through (d): (a) at least one substance that irritates the nasal and/or pharyngeal region; (b) at least one viscosity increasing agent that together with a required minimum quantity of an aqueous liquid forms a gel in an extract obtained from the dosage form, which gel can still be discerned after being introduced into an additional quantity of aqueous liquid; (c) at least one antagonist for the at least one active substance that is susceptible to abuse; and (d) at least one emetic.

### CROSS REFERENCE TO RELATED APPLICATIONS

This application is a continuation of international patent application no. PCT/EP2003/011784, filed Oct. 24, 2003, designating the United States of America, and published in German on May 6, 2004 as WO 2004/037259, the entire disclosure of which is incorporated herein by reference. Priority is claimed based on Federal Republic of Germany patent application no. DE 102 50 084.3, filed Oct. 25, 2002.

### BACKGROUND OF THE INVENTION

The present invention relates to an abuse-proofed dosage form which, apart from one or more active ingredients with potential for abuse, comprises two or more of the following components (a) through (d):
(a) at least one substance which irritates the nasal passages and/or pharynx;
(b) at least one viscosity-increasing agent, which, with the assistance of a necessary minimum quantity of an aqueous liquid, forms a gel with the extract obtained from the dosage form, which gel remains visually distinguishable when introduced into a further quantity of an aqueous liquid;
(c) at least one antagonist for the active ingredient or active ingredients with potential for abuse, and
(d) at least one emetic.

Many pharmaceutical active ingredients, in addition to having excellent activity in their appropriate application, also have potential for abuse, i.e. they can be used by an abuser to bring about effects other than the medical ones intended. Opiates, for example, which are highly active in combating severe to very severe pain, are frequently used by abusers to achieve a state of narcosis or euphoria.

Oral dosage forms which contain such active ingredients with potential for abuse do not usually give rise to the result desired by the abuser, even when taken in an abusively large quantity, because blood levels of the active ingredients increase only slowly. In order nevertheless to enable abuse, the corresponding dosage forms are comminuted, for example ground, by the abuser and administered, for example, by sniffing nasally. In another form of abuse, the active ingredient is extracted from the powder obtained by comminution of the dosage form using a preferably aqueous liquid and the resultant solution, optionally after being filtered through cotton wool or cellulose wadding, is administered parenterally, in particular intravenously. These forms of administration give rise to an accelerated rise in levels of the active ingredient, relative to oral administration, providing the abuser with the desired result.

### SUMMARY OF THE INVENTION

The object of the present invention was therefore to provide a dosage form for active ingredients with potential for abuse, which ensures the therapeutic action thereof on correct administration but does not have the action desired by the abuser when taken abusively.

This object has been achieved by the abuse-proofed dosage form according to the invention which, apart from one or more active ingredients with potential for abuse, comprises two or more of the following components (a) through (d):
(a) at least one substance which irritates the nasal passages and/or pharynx;
(b) at least one viscosity-increasing agent, which, with the assistance of a necessary minimum quantity of an aqueous liquid, forms a gel with the extract obtained from the dosage form, which gel remains visually distinguishable when introduced into a further quantity of an aqueous liquid;
(c) at least one antagonist for the active ingredient or active ingredients with potential for abuse, and
(d) at least one emetic.

Components (a) to (d) are additionally each individually suitable for abuse-proofing the dosage form according to the invention. Component (a) is accordingly preferably suitable for countering nasal and/or parenteral abuse, component (b) is preferably suitable for countering parenteral abuse, component (c) is preferably suitable for countering nasal and/or parenteral abuse and component (d) is preferably suitable for countering parenteral and/or oral and/or nasal abuse. The combination according to the invention of at least two of these above-stated components makes it possible to protect the dosage form according to the invention still more effectively from abuse.

In one embodiment, the dosage form according to the invention comprises three of components (a)-(d) in the abuse combination, preferably (a), (b) and (c) or (a), (b) and (d).

In a further embodiment, the dosage form according to the invention comprises all of components (a)-(d).

Pharmaceutical active ingredients with potential for abuse are known to persons skilled in the art, as are the quantities thereof to be used and processes for the production thereof, and may be present in the dosage form according to the invention as such, in the form of corresponding derivatives, in particular esters or ethers, or in each case in the form of corresponding physiologically acceptable compounds, in particular in the form of the salts or solvates thereof.

A combination of two or three of components (a), (b) and (d) is in particular suitable for preventing the abuse of a pharmaceutical active ingredient which is selected from the group consisting of opiates, opioids, tranquillizers, preferably benzodiazepines, stimulants and further narcotics.

In particular, a combination of two or three of components (a), (b) and (d) is suitable for preventing the abuse of opiates, opioids, tranquillizers and further narcotics, which are selected from the group consisting of N-{1-[2-(4-ethyl-5-oxo-2-tetrazolin-1-yl)ethyl]-4-methoxymethyl-4-piperidyl}propionanilide (alfentanil), 5,5-diallylbarbituric acid (allobarbital), allylprodine, alphaprodine, 8-chloro-1-methyl-6-phenyl-4H-[1,2,4]triazolo[4,3-a][1,4]-benzodiazepine (alprazolam), 2-diethylaminopropiophenone (amfepramone), (±)-α-methylphenethylamine (amphetamine), 2-(α-methylphenethylamino)-2-phenylacetonitrile (amphetaminil), 5-ethyl-5-isopentylbarbituric acid (amobarbital) anileridine, apocodeine, 5,5-diethylbarbituric acid (barbital), benzylmorphine, bezitramide, 7-bromo-5-(2-pyridyl)-1H-1,4-benzodiazepine-2(3H)-one (bromazepam), 2-bromo-4-(2-chlorophenyl)-9-methyl-6H-thieno[3,2-f][1,2,4]triazolo[4,3-a][1,4]diazepine (brotizolam), 17-cyclopropylmethyl-4,5α-epoxy-7α[(S)-1-hydroxy-1,2,2-trimethyl-propyl]-6-methoxy-6,14-endo-ethanomorphinan-3-ol (buprenorphine), 5-butyl-5-ethylbarbituric acid (butobarbital), butorphanol, (7-chloro-1,3-dihydro-1-methyl-2-oxo-5-phenyl-2H-1,4-benzodiazepin-3-yl) dimethylcarbamate (camazepam), (1S,2S)-2-amino-1-phenyl-1-propanol (cathine/D-norpseudoephedrine), 7-chloro-N-methyl-5-phenyl-3H-1,4-benzodiazepin-2-ylamine 4-oxide (chlordiazepoxide), 7-chloro-1-methyl-5-phenyl-1H-1,5-benzodiazepine-2,4(3H,5H)-dione (clobazam), 5-(2-chlorophenyl)-7-nitro-1H-1,4-benzodiazepin-2(3H)-one (clonazepam), clonitazene, 7-chloro-2,3-dihydro-2-oxo-5-phenyl-1H-1,4-benzodiazepine-3-carboxylic acid (clorazepate), 5-(2-chlorophenyl)-7-ethyl-1-methyl-1H-thieno[2,3-e][1,4]diazepin-2(3H)-one (clotiazepam), 10-chloro-11b-(2-chlorophenyl)-2,3,7,11b-tetrahydrooxazolo[3,2-d][1,4]benzodiazepin-6(5H)-one (cloxazolam), (−)-methyl-[3β-benzoyloxy-2β(1αH,5αH)-tropane carboxylate] (cocaine), 4,5α-epoxy-3-methoxy-17-methyl-7-morphinen-6α-ol (codeine), 5-(1-cyclohexenyl)-5-ethylbarbituric acid (cyclobarbital), cyclorphan, cyprenorphine, 7-chloro-5-(2-chlorophenyl)-1H-1,4-benzodiazepin-2(3H)-one (delorazepam), desomorphine, dextromoramide, (+)-(1-benzyl-3-dimethylamino-2-methyl-1-phenylpropyl) propionate (dextropropoxyphene), dezocine, diampromide, diamorphone, 7-chloro-1-methyl-5-phenyl-1H-1,4-benzodiazepin-2(3H)-one (diazepam), 4,5α-epoxy-3-methoxy-17-methyl-6α-morphinanol (dihydrocodeine), 4,5α-epoxy-17-methyl-3,6a-morphinandiol (dihydromorphine), dimenoxadol, dimephetamol, dimethylthiambutene, dioxaphetyl butyrate, dipipanone, (6aR,10aR)-6,6,9-trimethyl-3-pentyl-6a,7,8,10a-tetrahydro-6H-benzo[c]chromen-1-ol (dronabinol), eptazocine, 8-chloro-6-phenyl-4H-[1,2,4]triazolo[4,3-a][1,4]benzodiazepine (estazolam), ethoheptazine, ethylmethylthiambutene, ethyl [7-chloro-5-(2-fluorophenyl)-2,3-dihydro-2-oxo-1H-1,4-benzodiazepine-3-carboxylate] (ethyl loflazepate), 4,5α-epoxy-3-ethoxy-17-methyl-7-morphinen-6α-ol (ethylmorphine), etonitazene, 4,5α-epoxy-7α-(1-hydroxy-1-methylbutyl)-6-methoxy-17-methyl-6,14-endo-etheno-morphinan-3-ol (etorphine), N-ethyl-3-phenyl-8,9,10-trinorbornan-2-ylamine (fencamfamine), 7-[2-(α-methylphenethylamino)ethyl]-theophylline) (fenethylline), 3-(α-methylphenethylamino)propionitrile (fenproporex), N-(1-phenethyl-4-piperidyl)propionanilide (fentanyl), 7-chloro-5-(2-fluorophenyl)-1-methyl-1H-1,4-benzodiazepin-2(3H)-one (fludiazepam), 5-(2-fluorophenyl)-1-methyl-7-nitro-1H-1,4-benzodiazepin-2(3H)-one (flunitrazepam), 7-chloro-1-(2-diethylaminoethyl)-5-(2-fluorophenyl)-1H-1,4-benzodiazepin-2(3H)-one (flurazepam), 7-chloro-5-phenyl-1-(2,2,2-trifluoroethyl)-1H-1,4-benzodiazepin-2(3H)-one (halazepam), 10-bromo-11b-(2-fluorophenyl)-2,3,7,11b-tetrahydro[1,3]oxazolo[3,2-d][1,4]benzodiazepin-6(5H)-one (haloxazolam), heroin, 4,5α-epoxy-3-methoxy-17-methyl-6-morphinanone (hydrocodone), 4,5α-epoxy-3-hydroxy-17-methyl-6-morphinanone (hydromorphone), hydroxypethidine, isomethadone, hydroxymethylmorphinan, 11-chloro-8,12b-dihydro-2,8-dimethyl-12b-phenyl-4H-[1,3]oxazino[3,2-d][1,4]benzodiazepine-4,7(6H)-dione (ketazolam), 1-[4-(3-hydroxyphenyl)-1-methyl-4-piperidyl]-1-propanone (ketobemidone), (3S,6S)-6-dimethylamino-4,4-diphenylheptan-3-yl acetate (levacetylmethadol (LAAM)), (−)-6-dimethylamino-4,4-diphenol-3-heptanone (levomethadone), (−)-17-methyl-3-morphinanol (levorphanol), levophenacylmorphane, lofentanil, 6-(2-chlorophenyl)-2-(4-methyl-1-piperazinylmethylene)-8-nitro-2H-imidazo[1,2-a][1,4]-benzodiazepin-1(4H)-one (loprazolam), 7-chloro-5-(2-chlorophenyl)-3-hydroxy-1H-1,4-benzodiazepin-2(3H)-one (lorazepam), 7-chloro-5-(2-chlorophenyl)-3-hydroxy-1-methyl-1H-1,4-benzodiazepin-2(3H)-one (lormetazepam), 5-(4-chlorophenyl)-2,5-dihydro-3H-imidazo[2,1-a]isoindol-5-ol (mazindol), 7-chloro-2,3-dihydro-1-methyl-5-phenyl-1H-1,4-benzodiazepine (medazepam), N-(3-chloropropyl)-α-methylphenethylamine (mefenorex), meperidine, 2-methyl-2-propyltrimethylene dicarbamate (meprobamate), meptazinol, metazocine, methylmorphine, N,α-dimethylphenethylamine (metamphetamine), (±)-6-dimethylamino-4,4-diphenol-3-heptanone (methadone), 2-methyl-3-o-tolyl-4(3H)-quinazolinone (methaqualone), methyl [2-phenyl-2-(2-piperidyl)acetate] (methylphenidate), 5-ethyl-1-methyl-5-phenylbarbituric acid (methylphenobarbital), 3,3-diethyl-5-methyl-2,4-piperidinedione (methyprylon), metopon, 8-chloro-6-(2-fluorophenyl)-1-methyl-4H-imidazo[1,5-a][1,4]benzodiazepine (midazolam), 2-(benzhydrylsulfinyl)acetamide (modafinil), 4,5α-epoxy-17-methyl-7-morphinen-3,6α-diol (morphine), myrophine, (±)-trans-3-(1,1-dimethylheptyl)-7,8,10,10α-tetrahydro-1-hydroxy-6,6-dimethyl-6H-dibenzo-[b,d]pyran-9(6αH)-one (nabilone), nalbuphene, nalorphine, narceine, nicomorphine, 1-methyl-7-nitro-5-phenyl-1H-1,4-benzodiazepin-2(3H)-one (nimetazepam), 7-nitro-5-phenyl-1H-1,4-benzodiazepin-2(3H)-one (nitrazepam), 7-chloro-5-phenyl-1H-1,4-benzodiazepin-2(3H)-one (nordazepam), norlevorphanol, 6-dimethylamino-4,4-diphenyl-3-hexanone (normethadone), normorphine, norpipanone, the exudation from plants belonging to the species Papaver somniferum (opium), 7-chloro-3-hydroxy-5-phenyl-1H-1,4-benzodiazepin-2(3H)-one (oxazepam), (cis-trans)-10-chloro-2,3,7,11b-tetrahydro-2-methyl-11b-phenyloxazolo[3,2-d][1,4]benzodiazepin-6-(5H)-one (oxazolam), 4,5α-epoxy-14-hydroxy-3-methoxy-17-methyl-6-morphinanone (oxycodone), oxymorphone, plants and parts of plants belonging to the species Papaver somniferum (including the subspecies setigerum) (Papaver somniferum), papaveretum, 2-imino-5-phenyl-4-oxazolidinone (pernoline), 1,2,3,4,5,6-hexahydro-6,11-dimethyl-3-(3-methyl-2-butenyl)-2,6-methano-3-benzazocin-8-ol (pentazocine), 5-ethyl-5-(1-methylbutyl)-barbituric acid (pentobarbital), ethyl (1-methyl-4-phenyl-4-piperidinecarboxylate) (pethidine), phenadoxone, phenomorphane, phenazocine, phenoperidine, piminodine, pholcodeine, 3-methyl-2-phenylmorpholine (phenmetrazine), 5-ethyl-5-phenylbarbituric acid (phenobarbital), α,α-dimethylphenethylamine (phentermine), 7-chloro-5-phenyl-1-(2-propynyl)-1H-1,4-benzodiazepin-2(3H)-one (pinazepam), α-(2-piperidyl)benzhydryl alcohol (pipradrol), 1′-(3-cyano-3,3-diphenylpropyl)[1,4′-bipiperidine]-4′-carboxamide (piritramide), 7-chloro-1-(cyclopropylmethyl)-5-phenyl-1H-1,4-benzodiazepin-2(3H)-one (prazepam), profadol, proheptazine, promedol, properidine, propoxyphene, N-(1-methyl-2-piperidinoethyl)-N-(2-pyridyl)propionamide, methyl {3-[4-methoxycarbonyl-4-(N-phenylpropanamido)piperidino]propanoate} (remifentanil), 5-sec-butyl-5-ethylbarbituric acid (secbutabarbital), 5-allyl-5-(1-methylbutyl)-barbituric acid (secobarbital), N-{4-methoxymethyl-1-[2-(2-thienyl)ethyl]-4-piperidyl}propionanilide (sufentanil), 7-chloro-2-hydroxy-methyl-5-phenyl-1H-1,4-benzodiazepin-2(3H)-one (temazepam), 7-chloro-5-(1-cyclohexenyl)-1-methyl-1H-1,4-benzodiazepin-2(3H)-one (tetrazepam), ethyl (2-dimethylamino-1-phenyl-3-cyclohexene-1-carboxylate) (tilidine, cis and trans)), tramadol, 8-chloro-6-(2-chlorophenyl)-1-methyl-4H-[1,2,4]triazolo[4,3-a][1,4]benzodiazepine (triazolam), 5-(1-methylbutyl)-5-vinylbarbituric acid (vinylbital), (1R*,2R*)-3-(3-dimethylamino-1-ethyl-2-methyl-propyl)-phenol, (1R,2R,4S)-2-(dimethylamino)methyl-4-(p-fluorobenzyloxy)-1-(m-methoxyphenyl)cyclohexanol, in each case optionally in the form of corresponding stereoisomeric compounds and corresponding derivatives, in particular esters or ethers, and respective physiologically acceptable compounds, in particular salts and solvates.

If the abuse-proofing combination comprises component (c) for countering abuse, the combination is in particular suitable for preventing abuse of a pharmaceutical active ingredient which is selected from the group consisting of opiates, opioids, stimulants and further narcotics.

One particularly suitable combination is one which comprises component (c) for preventing the abuse of opiates, opioids and further narcotics which are selected from the group consisting of N-{1-[2-(4-ethyl-5-oxo-2-tetrazolin-1-yl)ethyl]-4-methoxymethyl-4-piperidyl}propionanilide (alfentanil), allylprodine, alphaprodine, 2-diethylaminopropiophenone (amfepramone), (±)-α-methylphenethylamine (amphetamine), 2-(α-methylphenethylamino)-2-phenylacetonitrile (amphetaminil), anileridine, apocodeine, benzylmorphine, bezitramide, 17-cyclopropylmethyl-4,5α-epoxy-7α[(S)-1-hydroxy-1,2,2-trimethyl-propyl]-6-methoxy-6,14-endo-ethanomorphinan-3-ol (buprenorphine), butorphanol, (1S,2S)-2-amino-1-phenyl-1-propanol (cathine/D-norpseudoephedrine), clonitazene, (−)-methyl-[3β-benzoyloxy-2β(1αH,5αH)-tropane carboxylate] (cocaine), 4,5α-epoxy-3-methoxy-17-methyl-7-morphinen-6α-ol (codeine), cyclorphan, cyprenorphine, desomorphine, dextromoramide, (+)-(1-benzyl-3-dimethylamino-2-methyl-1-phenylpropyl) propionate (dextropropoxyphene), dezocine, diampromide, diamorphone, 4,5α-epoxy-3-methoxy-17-methyl-6α-morphinanol (dihydrocodeine), 4,5α-epoxy-17-methyl-3,6a-morphinandiol (dihydromorphine), dimenoxadol, dimephetamol, dimethylthiambutene, dioxaphetyl butyrate, dipipanone, (6aR,10aR)-6,6,9-trimethyl-3-pentyl-6a,7,8,10a-tetrahydro-6H-benzo[c]chromen-1-ol (dronabinol), eptazocine, ethoheptazine, ethylmethylthiambutene, 4,5α-epoxy-3-ethoxy-17-methyl-7-morphinen-6α-ol (ethylmorphine), etonitazene, 4,5α-epoxy-7α-(1-hydroxy-1-methylbutyl)-6-methoxy-17-methyl-6,14-endo-etheno-morphinan-3-ol (etorphine), N-ethyl-3-phenyl-8,9,10-trinorbornan-2-ylamine (fencamfamine), 7-[2-(α-methylphenethylamino)ethyl]-theophylline) (fenethylline), 3-(α-methylphenethylamino)propionitrile (fenproporex), N-(1-phenethyl-4-piperidyl)propionanilide (fentanyl), heroin, 4,5α-epoxy-3-methoxy-17-methyl-6-morphinanone (hydrocodone), 4,5α-epoxy-3-hydroxy-17-methyl-6-morphinanone (hydromorphone), hydroxypethidine, isomethadone, hydroxymethylmorphinan, 1-[4-(3-hydroxyphenyl)-1-methyl-4-piperidyl]-1-propanone (ketobemidone), (3S,6S)-6-dimethylamino-4,4-diphenylheptan-3-yl acetate (levacetylmethadol (LAAM)), (−)-6-dimethylamino-4,4-diphenol-3-heptanone (levomethadone), (−)-17-methyl-3-morphinanol (levorphanol), levophenacylmorphane, lofentanil, 5-(4-chlorophenyl)-2,5-dihydro-3H-imidazo[2,1-a]isoindol-5-ol (mazindol), N-(3-chloropropyl)-α-methylphenethylamine (mefenorex), meperidine, meptazinol, metazocine, methylmorphine, N,α-dimethylphenethylamine (metamphetamine), (±)-6-dimethylamino-4,4-diphenol-3-heptanone (methadone), methyl [2-phenyl-2-(2-piperidyl)acetate] (methylphenidate), 3,3-diethyl-5-methyl-2,4-piperidinedione (methyprylon), 2-(benzhydrylsulfinyl)acetamide (modafinil), 4,5α-epoxy-17-methyl-7-morphinen-3,6α-diol (morphine), myrophine, (±)-trans-3-(1,1-dimethylheptyl)-7,8,10,10α-tetrahydro-1-hydroxy-6,6-dimethyl-6H-dibenzo-[b,d]pyran-9(6αH)-one (nabilone), nalbuphene, narceine, nicomorphine, norlevorphanol, 6-dimethylamino-4,4-diphenyl-3-hexanone (normethadone), normorphine, norpipanone, the exudation from plants belonging to the species Papaver somniferum (opium), 4,5α-epoxy-14-hydroxy-3-methoxy-17-methyl-6-morphinanone (oxycodone), oxymorphone, plants and parts of plants belonging to the species Papaver somniferum (including the subspecies setigerum) (Papaver somniferum), papaveretum, 2-imino-5-phenyl-4-oxazolidinone (pernoline), 1,2,3,4,5,6-hexahydro-6,11-dimethyl-3-(3-methyl-2-butenyl)-2,6-methano-3-benzazocin-8-ol (pentazocine), ethyl (1-methyl-4-phenyl-4-piperidinecarboxylate) (pethidine), phenadoxone, phenomorphane, phenazocine, phenoperidine, piminodine, pholcodeine, 3-methyl-2-phenylmorpholine (phenmetrazine), α,α-dimethylphenethylamine (phentermine), α-(2-piperidyl)benzhydryl alcohol (pipradrol), 1′-(3-cyano-3,3-diphenylpropyl)[1,4′-bipiperidine]-4′-carboxamide (piritramide), profadol, proheptazine, promedol, properidine, propoxyphene, N-(1-methyl-2-piperidinoethyl)-N-(2-pyridyl)propionamide, methyl {3-[4-methoxycarbonyl-4-(N-phenylpropanamido)piperidino]propanoate} (remifentanil), N-{4-methoxymethyl-1-[2-(2-thienyl)ethyl]-4-piperidyl}propionanilide (sufentanil), ethyl (2-dimethylamino-1-phenyl-3-cyclohexene-1-carboxylate) (tilidine, cis and trans)), tramadol, (1R*,2R*)-3-(3-dimethylamino-1-ethyl-2-methyl-propyl)-phenol, (1R,2R,4S)-2-(dimethylamino)methyl-4-(p-fluorobenzyloxy)-1-(m-methoxyphenyl)cyclohexanol, in each case optionally in the form of corresponding stereoisomeric compounds and corresponding derivatives, in particular esters or ethers, and in each case physiologically acceptable compounds, in particular salts and solvates.

The compounds (1R*,2R*)-3-(3-dimethylamino-1-ethyl-2-methyl-propyl)-phenol and (1R,2R,4S)-2-(dimethylamino)methyl-4-(p-fluorobenzyl-oxy)-1-(m-methoxyphenyl)cyclohexanol, the physiologically acceptable compounds thereof, in particular the hydrochlorides thereof and processes for the production thereof are respectively known, for example, from U.S. Pat. No. 6,248,737 (=EP 693,475) and U.S. Pat. No. 5,801,201 (=EP 780,369), the disclosures of which are incorporated herein by reference.

The dosage form according to the invention containing a combination of at least two of components (a) through (d) is also suitable for preventing abuse of stimulants, preferably those selected from the group consisting of amphetamine, norpseudoephedrine, methylphenidate and corresponding physiologically acceptable compounds thereof, in particular the bases, salts and solvates thereof.

If the combination for abuse-proofing the dosage form according to the invention comprises component (a), substances which irritate the nasal passages and/or pharynx which may be considered according to the invention are any substances which, when administered via the nasal passages and/or pharynx, bring about a physical reaction which is either so unpleasant for the abuser that he/she does not wish to or cannot continue administration, for example burning, or physiologically counteracts taking of the corresponding active ingredient, for example due to increased nasal secretion or sneezing. It has additionally been found that these substances which irritate the nasal passages and/or pharynx conventionally also bring about a very unpleasant sensation or even unbearable pain when administered parenterally, in particular intravenously, such that the abuser does not wish to or cannot continue taking the substance.

Particularly suitable substances which irritate the nasal passages and/or pharynx are those which cause burning, itching, an urge to sneeze, increased formation of secretions or a combination of at least two of these stimuli. Appropriate substances and the quantities thereof which are conventionally to be used are known per se to those skilled in the art or may be identified by simple preliminary testing.

The component (a) substance which irritates the nasal passages and/or pharynx is preferably based on one or more constituents or one or more plant parts of at least one hot substance drug. Corresponding hot substance drugs are known per se to those skilled in the art and are described, for example, in “Pharmazeutische Biologie—Drogen und ihre Inhaltsstoffe” by Prof. Dr. Hildebert Wagner, 2nd., revised edition, Gustav Fischer Verlag, Stuttgart-New York, 1982, pages 82 et seq. The corresponding description is hereby incorporated by reference and is deemed to be part of the disclosure. The dosage form according to the invention may preferably contain the plant parts of the corresponding hot substance drugs in a quantity of 0.01 to 30 wt. %, particularly preferably of 0.1 to 0.5 wt. %, in each case relative to the total weight of the dosage unit. If one or more constituents of corresponding hot substance drugs are used, the quantity thereof in a dosage unit according to the invention preferably is from 0.001 to 0.005 wt. %, relative to the total weight of the dosage unit.

As used herein, the term “dosage unit” is taken to mean a separate or separable administration unit, such as for example a tablet or a capsule.

The dosage form according to the invention preferably comprises as component (a) one or more constituents of at least one hot substance drug selected from the group consisting of Allii sativi bulbus (garlic), Asari rhizoma cum herba (Asarum root and leaves), Calami rhizoma (calamus root), Capsici fructus (capsicum), Capsici fructus acer (cayenne pepper), Curcumae longae rhizoma (turmeric root), Curcumae xanthorrhizae rhizoma (Javanese turmeric root), Galangae rhizoma (galangal root), Myristicae semen (nutmeg), Piperis nigri fructus (black pepper), Sinapis albae semen (white mustard seed), Sinapis nigri semen (black mustard seed), Zedoariae rhizoma (zedoary root) and Zingiberis rhizoma (ginger root), particularly preferably from the group consisting of Capsici fructus (capsicum), Capsici fructus acer (cayenne pepper) and Piperis nigri fructus (black pepper).

The constituents of the hot substance drugs preferably comprise o-methoxy(methyl)phenol compounds, acid amide compounds, mustard oils or sulfide compounds or compounds derived therefrom.

The constituent of the hot substance drugs is particularly preferably selected from the group consisting of myristicin, elemicin, isoeugenol, β-asarone, safrole, gingerols, xanthorrhizol, capsaicinoids, preferably capsaicin, piperine, preferably trans-piperine, glucosinolates, preferably based on non-volatile mustard oils, particularly preferably based on p-hydroxybenzyl mustard oil, methylmercapto mustard oil or methylsulfonyl mustard oil, and compounds derived from these constituents.

Another option for additionally countering abuse of the dosage form according to the invention involves adding thereto at least one viscosity-increasing agent as an additional abuse-preventing component (b), which, with the assistance of a necessary minimum quantity of an aqueous liquid, forms a gel with the extract obtained from the dosage form, which gel remains visually distinguishable when introduced into a further quantity of an aqueous liquid

For the purposes of the present invention, visually distinguishable means that the active ingredient-containing gel formed with the assistance of a necessary minimum quantity of aqueous liquid, when introduced, preferably with a hypodermic needle, into a further quantity of aqueous liquid at 37° C., remains substantially insoluble and cohesive and cannot straightforwardly be dispersed in such a manner that it can safely be administered parenterally, in particular intravenously. The duration of the visual distinguishability is preferably at least one minute.

The increased viscosity of the extract makes it more difficult or even impossible for it to be passed through a needle or injected. It also means that when the resultant extract is introduced into a further quantity of aqueous liquid, for example by injection into blood, a largely cohesive thread is initially obtained which, while it may be broken up into smaller fragments by mechanical action, it cannot be dispersed or even dissolved in such a manner that it may safely be administered parenterally, in particular intravenously. In combination with component (a) and/or (d), this additionally leads to unpleasant burning and/or vomiting.

Intravenous administration of such an extract would most probably result in obstruction of blood vessels, associated with serious embolism or even death of the abuser.

In order to verify whether a viscosity-increasing agent is suitable as component (b) for use in the dosage form according to the invention, said agent is first formulated in a corresponding dosage form, the resultant dosage form is comminuted, preferably ground, and extracted with 10 ml of water at a temperature of 25° C. If a gel is formed which meets the above-stated conditions, the corresponding viscosity-increasing agent is suitable for the production of a dosage form according to the invention.

If abuse-proofing is provided in the dosage form according to the invention by a combination containing component (b), one or more viscosity-increasing agents are preferably used which are selected from the group consisting of microcrystalline cellulose with 11 wt. % carboxymethylcellulose sodium (Avicel® RC 591), carboxymethylcellulose sodium (Blanose®, CMC-Na C300P®, Frimulsion BLC-5®, Tylose C300 P®), polyacrylic acid (Carbopol® 980 NF, Carbopol® 981), locust bean flour (Cesagum® LA-200, Cesagum® LID/150, Cesagum® LN-1), citrus pectin (Cesapectin® HM Medium Rapid Set), waxy maize starch (C*Gel 04201®), sodium alginate (Frimulsion ALG (E401)®), guar flour (Frimulsion BM®, Polygum 26/1-75®), iota-carrageenan (Frimulsion D021®), karaya gum, gellan gum (Kelcogel F®, Kelcogel LT100®), galactomannan (Meyprogat 150®), tara stone flour (Polygum 43/1®), propylene glycol alginate (Protanal-Ester SD-LB®), apple pectin, lemon peel pectin, sodium hyaluronate, tragacanth, tara gum (Vidogum SP 200®), fermented polysaccharide welan gum (K1A96), xanthan gum (Xantural 180®). The names stated in brackets are the trade names by which the materials are known commercially. In general, a quantity of 0.1 to 5 wt. % of the stated viscosity-increasing agent(s) is sufficient to fulfil the above-stated conditions.

The component (b) viscosity-increasing agents, where provided, are preferably present in the dosage form according to the invention in quantities of ≧5 mg per dosage unit, i.e. per administration unit.

In a particularly preferred embodiment of the present invention, the viscosity-increasing agents used as component (b) in the abuse-proofing combination are those which, on extraction from the dosage form with the necessary minimum quantity of aqueous liquid, form a gel which encloses air bubbles. The resultant gels are distinguished by a turbid appearance, which provides the potential abuser with an additional optical warning and discourages him/her from administering the gel parenterally.

Surprisingly, it is possible to combine the active ingredients and at least the viscosity-increasing agents in the dosage form according to the invention without spatial separation from one another, without there being any impairment of release of the active ingredient from the correctly administered dosage form relative to a corresponding dosage form which does not comprise the viscosity-increasing agent.

Obviously, however, as described below, it is also possible to combine the viscosity-increasing agents and the active ingredients in the dosage form in a mutually spatially separate arrangement.

The dosage form according to the invention may moreover comprise component (c) in the abuse-proofing combination, namely one or more antagonists for the active ingredient or active ingredients with potential for abuse, wherein the quantity of antagonist is preferably spatially separate from the active ingredient and component (a) and/or (b) and, when correctly used, should not exert any effect.

Suitable antagonists for preventing abuse of the active ingredients are known per se to the person skilled in the art and may be present in the dosage form according to the invention as such or in the form of corresponding derivatives, in particular esters or ethers, or respective corresponding physiologically acceptable compounds, in particular in the form of the salts or solvates thereof.

If the active ingredient present in the dosage form is an opiate or an opioid, the antagonist used is preferably an antagonist selected from the group consisting of naloxone, naltrexone, nalmefene, nalide, nalmexone, nalorphine or naluphine, in each case optionally in the form of a corresponding physiologically acceptable compound, in particular in the form of a base, a salt or solvate. The corresponding antagonists, where addition of component (c) is provided, are preferably used in a quantity of ≧10 mg, particularly preferably in a quantity of 10 to 100 mg, very particularly preferably in a quantity of 10 to 50 mg per dosage form, i.e. per administration unit.

If the dosage form according to the invention comprises a stimulant as the active ingredient, the antagonist is preferably a neuroleptic, preferably selected from the group consisting of haloperidol, promethazine, fluphenazine, perphenazine, levomepromazine, thioridazine, perazine, chlorpromazine, chlorprothixine, zuclopentixol, flupentixol, prothipendyl, zotepine, benperidol, pipamperone, melperone and bromperidol. The dosage form according to the invention preferably comprises these antagonists in a conventional therapeutic dose known to the person skilled in the art, particularly preferably in a quantity of twice to four times the conventional dose per administration unit.

If the combination for abuse-proofing the dosage form according to the invention comprises component (d), it may comprise at least one emetic, which is preferably present in a spatially separate arrangement from the optionally present component (a) and/or (b) and the active ingredient and, when correctly used, is intended not to exert its effect in the body.

Suitable emetics for preventing abuse of the active ingredients are known to persons skilled in the art and may be present in the dosage form according to the invention as such or in the form of corresponding derivatives, in particular esters or ethers, or in the form of the respective corresponding physiologically acceptable compounds, in particular in the form of the salts or solvates thereof.

If the abuse-proofing combination contains component (d), an emetic based on one or more constituents of ipecacuanha (ipecac) root, preferably based on the constituent emetine, is preferably considered for use in the dosage form according to the invention, as are, for example, described in “Pharmazeutische Biologie—Drogen und ihre Inhaltsstoffe” by Prof. Dr. Hildebert Wagner, 2nd, revised edition, Gustav Fischer Verlag, Stuttgart, New York, 1982. The corresponding literature description is hereby incorporated by reference and is deemed to be part of the disclosure.

The dosage form according to the invention may preferably comprise the emetic emetine as component (d), preferably in a quantity of ≧10 mg, particularly preferably of ≧20 mg and very particularly preferably in a quantity of ≧40 mg per dosage form, i.e. administration unit.

Apomorphine may likewise preferably be used as an emetic for abuse-proofing according to the invention, preferably in a quantity of preferably ≧3 mg, particularly preferably of ≧5 mg and very particularly preferably of ≧7 mg per administration unit.

The dosage form according to the invention may be formulated in many different ways using conventional methods known to the person skilled in the art. Methods for formulating the dosage form are known to the person skilled in the art, for example from “Coated Pharmaceutical Dosage Forms—Fundamentals, Manufacturing Techniques, Biopharmaceutical Aspects, Test Methods and Raw Materials” by Kurt H. Bauer, K. Lehmann, Hermann P. Osterwald, Rothgang, Gerhart, 1st edition, 1998, Medpharm Scientific Publishers. The corresponding description is hereby incorporated by reference and is deemed to be part of the disclosure.

The dosage forms according to the invention are preferably suitable for oral administration. In a preferred embodiment, the dosage form according to the invention assumes the form of a tablet, a capsule or the form of an oral osmotic therapeutic system (OROS).

One particularly straightforward way of formulating the dosage form according to the invention consists in mixing two or more of components (a)-(d) with the active ingredient or active ingredients and optionally physiologically acceptable auxiliary substances and packaging this mixture in a capsule or press-molding it to form a tablet, subject to compliance with tolerance limits with regard to components (c) and/or (d) in the event of correct oral administration. Care must be taken with this kind of formulation of the dosage form to ensure that components (c) and/or (d) are formulated in such a manner or incorporated in such small amounts that, in the event of correct administration, they are able to exert virtually no impairing effect on the patient or the activity of the active ingredient.

In a further preferred embodiment the dosage form according to the invention contains component (d) in an amount which is selected such that, in the event of correct oral administered, no negative action is caused. If, however, the intended dosage of the dosage form is exceeded inadvertently, in particular by children, or in the event of abuse, nausea or an inclination to vomit are produced. The particular quantity of component (d) which can still be tolerated by the patient in the event of correct oral administration may be determined by the person skilled in the art by simple preliminary testing.

Oral osmotic therapeutic systems and suitable materials and processes for the production thereof are known per se to the person skilled in the art, for example from U.S. Pat. Nos. 4,612,008; 4,765,989 and 4,783,337, the disclosures of which are incorporated by reference.

If, however, a combination containing components (c) and/or (d) for abuse-proofing the dosage form is provided, these components should preferably be used in sufficiently large amounts that, when abusively administered, they bring about an intense negative effect on the abuser. This is preferably achieved by spatial separation of at least the active ingredient or active ingredients from components (c) and/or (d), wherein the active ingredient or active ingredients is/are preferably present in at least one subunit (A) and components (c) and/or (d) is/are present in at least one subunit (B), and wherein, when the dosage form is correctly administered, components (c) and (d) do not exert their effect in the body.

If the dosage form according to the invention comprises both of components (c) and (d), these may each be present in the same or different subunits (B). Preferably, when present, both components (c) and (d) are present in one and the same subunit (B).

For the purposes of the present invention, subunits are solid formulations, which in each case, apart from conventional auxiliary substances known to the person skilled in the art, contain only the active ingredient(s) and optionally at least one of the optionally present components (a) and/or (b) or only the antagonist(s) and/or emetic(s) and optionally at least one of the optionally present components (a) and/or (b).

One substantial advantage of the separate formulation of active ingredients from components (c) and (d) in subunits (A) and (B) of the dosage form according to the invention is that, when correctly administered, components (c) and/or (d) are hardly released in the body or are only released in such small quantities that they exert no effect which impairs the patient or therapeutic success or, on passing through the patient's body, they are only liberated in locations where they cannot be sufficiently absorbed to be effective. Components (c) and/or (d) are preferably practically not released in the patient's body when the dosage form is correctly administered. The person skilled in the art will understand that the above-stated conditions may vary as a function of the particular components (c) and (d) used and of the formulation of the subunits or the dosage form. The optimum formulation for the particular dosage form may be determined by simple preliminary testing.

If a corresponding dosage form according to the invention comprising components (c) and/or (d) in subunits (B) is manipulated for the purpose of abusive taking of the active ingredient, e.g. by grinding and optionally extracting the powder thus obtained with a suitable extracting agent, in addition to the active ingredient and optionally (a) and/or (b), the particular component (c) and/or (d) is also obtained in a form in which it cannot easily be separated from the active ingredient, such that, on administration of the manipulated dosage form, in particular in the case of oral and/or parenteral administration, its action develops in the body and optionally one of component (c) and/or (d) additionally causes a corresponding negative effect on the abuser and so prevents abuse of the dosage form.

A dosage form according to the invention, in which the active ingredient or active ingredients is/are spatially separate from components (c) and (d), preferably by formulation in different subunits, may be formulated in many different ways, wherein the corresponding subunits may each be present in the dosage form according to the invention in any desired spatial arrangement relative to one another, provided that the above-stated conditions for the release of components (c) and/or (d) are fulfilled.

Those skilled in the art will understand that the optionally present component(s) (a) and/or (b) may preferably be formulated in the dosage form produced according to the invention both in the particular subunits (A) and (B) and in the form of independent subunits corresponding to subunits (A) and (B), provided that neither the abuse-proofing nor the active ingredient release in the event of correct administration is impaired by the nature of the formulation.

In a preferred embodiment of the dosage form according to the invention, both subunits (A) and (B) are present in multiparticulate form, wherein microtablets, microcapsules, micropellets, granules, spheroids, beads or pellets are preferred and the same form, i.e. size and shape, is selected for both subunit (A) and subunit (B), such that it is not possible to separate subunits (A) from (B) by mechanical selection. The multiparticulate forms are preferably of a size in the range from 0.1 to 3 mm, preferably of 0.5 to 2 mm.

The subunits (A) and (B) in multiparticulate form may also preferably be packaged in a capsule, suspended in a liquid or a gel or be press-molded to form a tablet, wherein final formulation in each case proceeds in such a manner that the subunits (A) and (B) are also retained in the resultant dosage form.

The respective multiparticulate subunits (A) and (B) of identical shape must also not be visually distinguishable from one another so that the abuser cannot separate them from one another by simple sorting. This may, for example, be achieved by the application of identical coatings which, apart from this disguising function, may also incorporate further functions, such as, for example, delayed release of one or more active ingredients or provision of a finish resistant to gastric juices on the particular subunits.

In a further preferred embodiment of the present invention, subunits (A) and (B) respectively are arranged in layers relative to one another. The layered subunits (A) and (B) are preferably arranged for this purpose vertically or horizontally relative to one another in the dosage form according to the invention, wherein in each case one or more layered subunits (A) and one or more layered subunits (B) may be present in the dosage form, such that, apart from the preferred layer sequences (A)-(B) or (A)-(B)-(A), any desired other layer sequences may be considered, optionally in combination with layers containing components (a) and/or (b).

Another preferred dosage form according to the invention is one in which subunit (B) forms a core which is completely enclosed by subunit (A), wherein an optionally swellable separation layer (C) may be present between said layers. Such a structure is preferably also suitable for the above-stated multiparticulate forms, wherein both subunits (A) and (B) and an optionally present separation layer (C) are formulated in one and the same multiparticulate form.

In a further preferred embodiment of the dosage form according to the invention, the subunit (A) forms a core, which is enclosed by subunit (B), wherein the surrounding subunit (B) comprises at least one channel which leads from the core to the surface of the dosage form.

The dosage form according to the invention may comprise, between one layer of the subunit (A) and one layer of the subunit (B), in each case one or more, preferably one, optionally swellable separation layer (C) which serves to separate subunit (A) spatially from (B). If the dosage form according to the invention comprises the layered subunits (A) and (B) and an optionally present separation layer (C) in an at least partially vertical or horizontal arrangement, the dosage form preferably assumes the form of a tablet, a co-extrudate or a laminate.

In one particularly preferred embodiment, the entirety of the free surface of subunit (B) and optionally at least part of the free surface of subunit(s) (A) and optionally at least part of the free surface of the optionally present separation layer(s) (C) may be coated with at least one barrier layer (D) which prevents release of component (c) or (d).

Another particularly preferred embodiment of the dosage form according to the invention comprises a vertical or horizontal arrangement of the layers of subunits (A) and (B) and at least one push layer (p) arranged therebetween, and optionally a separation layer (C), in which dosage form the entirety of the free surfaces of the layer structure consisting of subunits (A) and (B), the push layer and the optionally present separation layer (C) are provided with a semipermeable coating (E), which is permeable to a release medium, i.e. conventionally a physiological liquid, but substantially impermeable to the active ingredient and to component (c) and/or (d), and wherein this coating (E) comprises at least one opening for release of the active ingredient in the area of subunit (A).

A corresponding dosage form is known to those skilled in the art, for example under the name oral osmotic therapeutic system (OROS), as are suitable materials and methods for the production thereof, inter alia from U.S. Pat. Nos. 4,612,008; 4,765,989 and 4,783,337, the disclosures of which are incorporated herein by reference.

In a further preferred embodiment, the subunit (A) of the dosage form according to the invention is in the form of a tablet having a peripheral edge face and two main faces, and the edge face and optionally one of the two main faces is covered with a barrier layer (B) containing component (c) and/or (d).

Those skilled in the art will understand that the auxiliary substances of the subunit(s) (A) or (B) and of the optionally present separation layer(s) (C) and/or of the barrier layer(s) (D) used in formulating the dosage form according to the invention will vary as a function of the arrangement thereof in the dosage form according to the invention, the mode of administration and as a function of the particular active ingredient of the optionally present components (a) and/or (b) and of component (c) and/or (d). The materials which have the requisite properties are in each case known per se to persons skilled in the art.

If release of component (c) and/or (d) the emetic from subunit (B) of the dosage form according to the invention is prevented with the assistance of a cover, preferably a barrier layer, the subunit may consist of conventional materials known to persons skilled in the art.

If a corresponding barrier layer (D) is not provided to prevent release of component (c) and/or (d), the materials of the subunits should be selected such that release of the particular component (c) and/or (d) from subunit (B) is virtually ruled out. The materials which are stated below to be suitable for production of the barrier layer may preferably be used for this purpose.

Preferred materials include those which are selected from the group consisting of alkylcelluloses hydroxyalkylcelluloses, glucans, scleroglucans, mannans, xanthans, copolymers of poly[bis(p-carboxyphenoxy)propane and sebacic acid], preferably in a molar ratio of 20:80 (marketed under the name Polifeprosan 20®), carboxymethylcelluloses, cellulose ethers, cellulose esters, nitrocelluloses, polymers based on (meth)acrylic acid and the esters thereof, polyamides, polycarbonates, polyalkylenes, polyalkylene glycols, polyalkylene oxides, polyalkylene terephthalates, polyvinyl alcohols, polyvinyl ethers, polyvinyl esters, halogenated polyvinyls, polyglycolides, polysiloxanes and polyurethanes and the copolymers thereof.

Particularly suitable materials may be selected from the group consisting of methylcellulose, ethylcellulose, hydroxypropylcellulose, hydroxypropylmethylcellulose, hydroxybutylmethylcellulose, cellulose acetate, cellulose propionate (of low, medium or high molecular weight), cellulose acetate propionate, cellulose acetate butyrate, cellulose acetate phthalate, carboxymethylcellulose, cellulose triacetate, sodium cellulose sulfate, polymethyl methacrylate, polyethyl methacrylate, polybutyl methacrylate, polyisobutyl methacrylate, polyhexyl methacrylate, polyisodecyl methacrylate, polylauryl methacrylate, polyphenyl methacrylate, polymethyl acrylate, polyisopropyl acrylate, polyisobutyl acrylate, polyoctatdecyl acrylate, polyethylene, low density polyethylene, high density polyethylene, polypropylene, polyethylene glycol, polyethylene oxide, polyethylene terephthalate, polyvinyl alcohol, polyvinyl isobutyl ether, polyvinyl acetate and polyvinyl chloride.

Especially suitable copolymers may be selected from the group consisting of copolymers of butyl methacrylate and isobutyl methacrylate, copolymers of methyl vinyl ether and maleic acid of high molecular weight, copolymers of methyl vinyl ether and maleic acid monoethyl ester, copolymers of methyl vinyl ether and maleic anhydride and copolymers of vinyl alcohol and vinyl acetate.

Further materials which are particularly suitable for formulating the barrier layer include starch-filled polycaprolactone [WO98/20073], aliphatic polyesteramides [U.S. Pat. No. 6,344,535 (=DE 19 753 534), CA 2,317,747 (=DE 19 800 698), U.S. Pat. No. 5,928,739 (=EP 820,698)], aliphatic and aromatic polyester urethanes [U.S. Pat. No. 6,821,588 (=DE 19822979)], polyhydroxyalkanoates, in particular polyhydroxybutyrates, polyhydroxyvalerates, casein [U.S. Pat. No. 5,681,517 (=DE 4 309 528)], polylactides and copolylactides [U.S. Pat. No. 6,235,825 (=EP 980,894), the disclosures of which are incorporated herein by reference.

The above-stated materials may optionally be blended with further conventional auxiliary substances known to persons skilled in the art, preferably selected from the group consisting of glyceryl monostearate, semi-synthetic triglyceride derivatives, semi-synthetic glycerides, hydrogenated castor oil, glyceryl palmitostearate, glyceryl behenate, polyvinylpyrrolidone, gelatine, magnesium stearate, stearic acid, sodium stearate, talcum, sodium benzoate, boric acid and colloidal silica, fatty acids, substituted triglycerides, glycerides, polyoxyalkylene glycols and the derivatives thereof.

If the dosage form according to the invention comprises a separation layer (D), said layer, like the uncovered subunit (B), may preferably consist of the above-stated materials described for the barrier layer. Those skilled in the art will understand that release of the active ingredient or of component (c) and/or (d) from the particular subunit may be controlled by the thickness of the separation layer.

The dosage form produced according to the invention may comprise one or more active ingredients at least partially in delayed-release form, wherein delayed release may be achieved with the assistance of conventional materials and methods known to the person skilled in the art, for example by embedding the active ingredient in a delayed-release matrix or by the application of one or more delayed-release coatings. Active ingredient release must, however, be controlled such that the above-stated conditions are fulfilled in each case, for example that, in the event of correct administration of the dosage form, the active ingredient or active ingredients are virtually completely released before the optionally present component (c) and/or (d) can exert an impairing effect.

If the dosage form according to the invention is intended for oral administration, it may also preferably comprise a coating which is resistant to gastric juices and dissolves as a function of the pH value of the release environment. By means of this coating, it is possible to ensure that the dosage form according to the invention passes through the stomach undissolved and the active ingredient is only released in the intestines. The coating which is resistant to gastric juices preferably dissolves at a pH value of between 5 and 7.5.

Corresponding materials and methods for the controlled release of active ingredients and for the application of coatings which are resistant to gastric juices are known to the person skilled in the art, for example from “Coated Pharmaceutical Dosage Forms—Fundamentals, Manufacturing Techniques, Biopharmaceutical Aspects, Test Methods and Raw Materials” by Kurt H. Bauer, K. Lehmann, Hermann P. Osterwald, Rothgang, Gerhart, 1st edition, 1998, Medpharm Scientific Publishers. The corresponding literature description is hereby incorporated by reference and is deemed to be part of the disclosure.

The dosage forms according to the invention have the advantage that, by virtue of any desired combination of two or more of components (a)-(d), they are protected against any kind of abuse, preferably against oral, nasal and parenteral abuse, without there being any risk of harm to the patient being treated or a reduction in efficacy of the respective active ingredient in the event of correct administration. They may be produced simply and comparatively economically.

The following Examples are intended to illustrate the invention purely by way of example and without restricting its overall scope.

### EXAMPLES

### Example 1

### Matrix Tablets with the Following Composition Per Tablet

| | |
|---|---|
| (−)-(1R,2R)-3-(3-Dimethylamino-1-ethyl-2-methyl- | 100 mg |
| propyl)phenol hydrochloride | |
| Hydroxypropylmethylcellulose (Metolose 90 SH 100,000 | 70 mg |
| from Shinetsu), 100,000 mPa · s | |
| Xanthan, NF | 10 mg |
| Microcrystalline cellulose (Avicel PH 102 from FMC) | 113 mg |
| Cayenne pepper | 10 mg |
| Highly disperse silicon dioxide | 4 mg |
| Magnesium stearate | 3 mg |
| Total quantity | 310 mg |

were produced in the following manner in a batch size of 1000 tablets: All the constituents were weighed out and screened in Quadro Coil U10 screening machine using a screen size of 0.813 mm, mixed in a container mixer (Bohle LM 40) for 15 min±15 seconds at a rotational speed of 20±1 rpm and pressed on a Korsch EKO eccentric press to form biconvex tablets with a diameter of 10 mm, a radius of curvature of 8 mm and an average tablet weight of 310 mg.

One of the tablets was ground and shaken with 10 ml of water. A viscous, turbid suspension formed. Once the coarse, solid components of the suspension had settled out, the suspension was drawn up into a syringe with a 0.9 mm diameter needle, drawing up being made more difficult due to the viscosity. The drawn up extraction liquid was injected into water at 37° C. and threads, which did not mix with the water, with the diameter of the needle were clearly extruded. While the threads could be broken up by stirring, they were not dissolved and the thread fragments still remained visible to the naked eye after a few minutes. Were such an extract to be injected into blood vessels, vessel blockages would occur.

A crushed tablet was drawn out into a 10 cm long line and sniffed into the nose through a tube. After just the first cm, the nasal irritation was such as to produce an urgent need to remove the powder by sneezing and the remaining powder could no longer be sniffed up nasally. The nasal irritation subsided after sneezing and the irritation had largely disappeared after approx. 10 min. There was no urge to repeat the experience.

### Example 2

Matrix Tablets with the Following Composition Per Tablet

| | |
|---|---|
| (−)-(1R,2R)-3-(3-Dimethylamino-1-ethyl-2-methyl- | 100 mg |
| propyl)phenol hydrochloride | |
| Hydroxypropylmethylcellulose (Metolose 90 SH 100,000 | 40 mg |
| from Shinetsu), 100,000 mPa · s | |
| Xanthan, NF | 40 mg |
| Microcrystalline cellulose (Avicel PH 102 from FMC) | 113 mg |
| Cayenne pepper | 10 mg |
| Highly disperse silicon dioxide | 4 mg |
| Magnesium stearate | 3 mg |
| Total quantity | 310 mg |

were produced as described in Example 1.

One of the tablets was ground and shaken with 10 ml of water. A viscous, turbid suspension with enclosed air bubbles formed, the viscosity of which was greater than in Example 1; more air bubbles were also enclosed. Once the coarse, solid components of the suspension had settled out, the suspension was drawn up into a syringe with a 0.9 mm diameter needle, drawing up being made very much more difficult due to the viscosity. The drawn up extraction liquid was injected into water at 37° C. and threads, which did not mix with the water, with the diameter of the needle were clearly extruded. While the threads could be broken up by stirring, they were not dissolved and the thread fragments still remained visible to the naked eye after a few minutes. Were such an extract to be injected into blood vessels, vessel blockages would occur.

A crushed tablet was drawn out into a 10 cm long line and sniffed into the nose through a tube. After just the first cm, the nasal irritation was such as to produce an urgent need to remove the powder by sneezing and the remaining powder could no longer be sniffed up nasally. The nasal irritation subsided after sneezing and the irritation had largely disappeared after approx. 10 min. There was no urge to repeat the experience.

### Example 3

Matrix Tablets with the Following Composition Per Tablet

| | |
|---|---|
| (−)-(1R,2R)-3-(3-Dimethylamino-1-ethyl-2-methyl- | 100 mg |
| propyl)phenol hydrochloride | |
| Xanthan, NF | 80 mg |
| Microcrystalline cellulose (Avicel PH 102 from FMC) | 113 mg |
| Cayenne pepper | 10 mg |
| Highly disperse silicon dioxide | 4 mg |
| Magnesium stearate | 3 mg |
| Total quantity | 310 mg |

were produced as described in Example 1.

One of the tablets was ground and shaken with 10 ml of water. A viscous, turbid suspension with enclosed air bubbles formed, the viscosity of which was greater than in Example 1; still more air bubbles were also enclosed. Once the coarse, solid components of the suspension had settled out, the suspension was drawn up into a syringe with a 0.9 mm diameter needle, drawing up being made very much more difficult due to the viscosity. The drawn up extraction liquid was injected into water at 37° C. and threads, which did not mix with the water, with the diameter of the needle were clearly extruded. While the threads could be broken up by stirring, they were not dissolved and the thread fragments still remained visible to the naked eye after a few minutes. Were such an extract to be injected into blood vessels, vessel blockages would occur.

A crushed tablet was drawn out into a 10 cm long line and sniffed into the nose through a tube. After just the first cm, the nasal irritation was such as to produce an urgent need to remove the powder by sneezing and the remaining powder could no longer be sniffed up nasally. The nasal irritation subsided after sneezing and the irritation had largely disappeared after approx. 10 min. There was no urge to repeat the experience.

### Examples 4-7

Matrix Tablets with the Following Composition Per Tablet

| | Example | | | |
|---|---|---|---|---|
| | 4 | 5 | 6 | 7 |
| (−)-(1R,2R)-3-(3-Dimethylamino- | 100 mg | 100 mg | 100 mg | 100 mg |
| 1-ethyl-2-methyl-propyl)phenol | | | | |
| hydrochloride | | | | |
| Hydroxypropylmethylcellulose | 80 mg | 80 mg | 80 mg | 80 mg |
| (Metolose 90 SH 100,000 from | | | | |
| Shinetsu), 100,000 mPa · s | | | | |
| Carboxymethylcellulose (Tylose | 10 mg | | | |
| C300) | | | | |
| Carboxymethylcellulose (Tylose | | 10 mg | | |
| C600) | | | | |
| Hydroxyethylcellulose (Tylose | | | 10 mg | |
| H300) | | | | |
| Hydroxyethylcellulose (Tylose | | | | 10 mg |
| H4000) | | | | |
| Microcrystalline cellulose (Avicel | 113 mg | 113 mg | 113 mg | 113 mg |
| PH 102 from FMC) | | | | |
| Cayenne pepper | 10 mg | 10 mg | 10 mg | 10 mg |
| Highly disperse silicon dioxide | 4 mg | 4 mg | 4 mg | 4 mg |
| Magnesium stearate | 3 mg | 3 mg | 3 mg | 3 mg |
| Total quantity | 320 mg | 320 mg | 320 mg | 320 mg |

were produced as stated in Example 1.

The tablets were ground and shaken with 10 ml of water. A viscous, turbid suspension formed; air bubbles were also enclosed. Once the coarse, solid components of the suspension had settled out, the suspension was drawn up into a syringe with a 0.9 mm diameter needle, drawing up being made very much more difficult due to the viscosity. The drawn up extraction liquid was injected into water at 37° C. and threads, which did not mix with the water, with the diameter of the needle were clearly extruded. While the threads could be broken up by stirring, they were not dissolved and the thread fragments still remained visible to the naked eye after a few minutes. Were such an extract to be injected into blood vessels, vessel blockages would occur.

A crushed tablet was in each case drawn out into a 10 cm long line and sniffed into the nose through a tube. After just the first cm, the nasal irritation was such as to produce an urgent need to remove the powder by sneezing and the remaining powder could no longer be sniffed up nasally. The nasal irritation subsided after sneezing and the irritation had largely disappeared after 10 min. There was no urge to repeat the experience.

### Examples 8-13

Matrix Tablets with the Following Composition Per Tablet

| | Example | | | | | |
|---|---|---|---|---|---|---|
| | 8 | 9 | 10 | 11 | 12 | 13 |
| Morphine sulfate | 60 mg | 60 mg | 60 mg | 60 mg | 60 mg | 60 mg |
| pentahydrate | | | | | | |
| Hydroxypropylmethylcellulose | 60 mg | 60 mg | 60 mg | 60 mg | 60 mg | 60 mg |
| (Metolose 90 SH | | | | | | |
| 15,000 from Shinetsu), | | | | | | |
| 15,000 mPa · s | | | | | | |
| Xanthan, NF | 10 mg | 30 mg | | | | |
| Carboxymethylcellulose | | | 10 mg | | | |
| (Tylose C300) | | | | | | |
| Carboxymethylcellulose | | | | 10 mg | | |
| (Tylose C600) | | | | | | |
| Hydroxyethylcellulose | | | | | 10 mg | |
| (Tylose H300) | | | | | | |
| Hydroxyethylcellulose | | | | | | 10 mg |
| (Tylose H4000) | | | | | | |
| Microcrystalline cellulose | 112.9 mg | 112.9 mg | 112.9 mg | 112.95 mg | 112.95 mg | 112.95 mg |
| (Avicel PH 102 from | | | | | | |
| FMC) | | | | | | |
| Capsaicin, micronised | 0.1 mg | 0.1 mg | 0.1 mg | 0.05 mg | 0.05 mg | 0.05 mg |
| Highly disperse silicon | 4 mg | 4 mg | 4 mg | 4 mg | 4 mg | 4 mg |
| dioxide | | | | | | |
| Magnesium stearate | 3 mg | 3 mg | 3 mg | 3 mg | 3 mg | 3 mg |

were produced as stated in Example 1.

A tablet was ground and shaken with 10 ml of water. A viscous, turbid suspension formed; air bubbles were also enclosed. Once the coarse, solid components of the suspension had settled out, the suspension was drawn up into a syringe with a 0.9 mm diameter needle, drawing up being made very much more difficult due to the viscosity. The drawn up extraction liquid was injected into water at 37° C. and threads, which did not mix with the water, with the diameter of the needle were clearly extruded. While the threads could be broken up by stirring, they were not dissolved and the thread fragments still remained visible to the naked eye after a few minutes. Were such an extract to be injected into blood vessels, vessel blockages would occur.

A crushed tablet was in each case drawn out into a 10 cm long line and sniffed into the nose through a tube. After just the first cm, the nasal irritation was such as to produce an urgent need to remove the powder by sneezing and the remaining powder could no longer be sniffed up nasally. The nasal irritation subsided after sneezing and the irritation had largely disappeared after 10 min. There was no urge to repeat the experience.

### Examples 14-18

Capsules with the Following Composition of the Simple Powder Mixture Per Capsule (Size 4 Capsule)

| | Example | | | | |
|---|---|---|---|---|---|
| | 14 | 15 | 16 | 17 | 18 |
| Morphine sulfate | 20 mg | 20 mg | 20 mg | 20 mg | 20 mg |
| pentahydrate | | | | | |
| Xanthan, NF | 10 mg | | | | |
| Carboxymethylcellulose | | 10 mg | | | |
| (Tylose C300) | | | | | |
| Carboxymethylcellulose | | | 10 mg | | |
| (Tylose C600) | | | | | |
| Hydroxyethylcellulose | | | | 10 mg | |
| (Tylose H300) | | | | | |
| Hydroxyethylcellulose | | | | | 10 mg |
| (Tylose H4000) | | | | | |
| Microcrystalline | 63 mg | 63 mg | 63 mg | 63 mg | 63 mg |
| cellulose (Avicel | | | | | |
| PH102 from FMC) | | | | | |
| Cayenne pepper | 5 mg | 5 mg | 5 mg | 5 mg | 5 mg |
| Highly disperse silicon | 1 mg | 1 mg | 1 mg | 1 mg | 1 mg |
| dioxide | | | | | |
| Magnesium stearate | 1 mg | 1 mg | 1 mg | 1 mg | 1 mg |

The powder from the capsules was ground and shaken with 10 ml of water. A viscous, turbid suspension formed; air bubbles were also enclosed. Once the coarse, solid components of the suspension had settled out, the suspension was drawn up into a syringe with a 0.9 mm diameter needle, drawing up being made very much more difficult due to the viscosity. The drawn up extraction liquid was injected into water at 37° C. and threads, which did not mix with the water, with the diameter of the needle were clearly extruded. While the threads could be broken up by stirring, they were not dissolved and the thread fragments still remained visible to the naked eye after a few minutes. Were such an extract to be injected into blood vessels, vessel blockages would occur. The crushed powder was in each case drawn out into a 10 cm long line and sniffed into the nose through a tube. After just the first cm, the nasal irritation was such as to produce an urgent need to remove the powder by sneezing and the remaining powder could no longer be sniffed up nasally. The nasal irritation subsided after sneezing and the irritation had largely disappeared after approx. 10 min. There was no urge to repeat the experience.

The quantities indicated below relate in each case to the composition of a dosage form. A batch from a single production run comprises 1000 such dosage forms.

### Example 19

### Jacketed Tablets

Core

| | |
|---|---|
| Emetine | 50 mg |
| Hydrogenated castor oil (Cutina HR) | 50 mg |

Emetine and finely powdered hydrogenated castor oil were mixed and press-molded in a tablet press to form round, biconvex tables of a diameter of 6.5 mm.

Jacket

| | |
|---|---|
| Morphine sulfate pentahydrate | 60 mg |
| Methylhydroxypropylcellulose 100,000 mPa · s (Metolose | 100 mg |
| 90 SH 100,000, ShinEtsu) | |
| Microcrystalline cellulose (Avicel PH 102) | 155 mg |
| Lactose monohydrate | 165 mg |
| Cayenne pepper | 10 mg |
| Magnesium stearate | 5 mg |
| Colloidal silicon dioxide | 5 mg |

All the jacket constituents were mixed; approx. 250 mg of the mixture were placed in the tablet die in a tablet press with a tool for 13 mm biconvex tablets, the 6.5 mm core was inserted centrally, the remaining 250 mg of jacket mixture were added and the jacket was pressed around the core.

### Example 20

### Jacketed Tablets

Core

| | |
|---|---|
| Emetine | 50 mg |
| Hydrogenated castor oil (Cutina HR) | 50 mg |

Emetine and finely powdered hydrogenated castor oil were mixed and press-molded in a tablet press to form round, biconvex tables of a diameter of 6.5 mm.

Jacket

| | |
|---|---|
| Oxycodone hydrochloride | 30 mg |
| Spray-dried lactose | 290 mg |
| Eudragit RSPM | 70 mg |
| Stearyl alcohol | 115 mg |
| Cayenne pepper | 10 mg |
| Magnesium stearate | 5 mg |
| Talcum | 10 mg |

Oxycodone hydrochloride, spray-dried lactose and Eudragit RSPM were intimately mixed together for approx. 5 min in a suitable mixer. During mixing, the mixture was granulated with such a quantity of purified water that a moist, granulated mass was formed. The resultant granular product was dried in a fluidized bed at 60° C. and passed through a 2.5 mm screen. The granular product was then dried again as described above and passed through a 1.5 mm screen. The stearyl alcohol was melted at 60-70° C. and added to the granular product in a mixer. After cooling, the mass was pressed together with cayenne pepper, magnesium stearate and talcum through a 1.5 mm screen. From the resultant granular product, approx. 265 mg of the mixture were placed in the tablet die in a tablet press with a tool for 13 mm biconvex tablets, the 6.5 mm core was inserted centrally, the remaining 265 mg of the jacket mixture were added and the jacket was pressed around the core.

### Example 21

### Jacketed Tablets

Core

| | |
|---|---|
| Naltrexone hydrochloride | 50 mg |
| Spray-dried lactose | 46 mg |
| Magnesium stearate | 2 mg |
| Colloidal silicon dioxide | 2 mg |

All the constituents were mixed and press-molded in a tablet press to form round, biconvex tablets of a diameter of 6.5 mm.

Coating on Core

| | |
|---|---|
| Cellulose acetate with 39.8% acetate | 9.5 mg |
| Macrogol 3350 | 0.5 mg |

The coating constituents were dissolved in an acetone-water mixture (95:5 parts by weight) and sprayed onto the cores.

Jacket

| | |
|---|---|
| Morphine sulfate pentahydrate | 60 mg |
| Methylhydroxypropylcellulose 100,000 mPa · s | 100 mg |
| (Metolose 90 SH 100,000, ShinEtsu) | |
| Microcrystalline cellulose (Avicel PH 102) | 165 mg |
| Lactose monohydrate | 155 mg |
| Cayenne pepper | 10 mg |
| Magnesium stearate | 5 mg |
| Colloidal silicon dioxide | 5 mg |

All the jacket constituents were mixed; approx. 250 mg of the mixture were placed in the tablet die in a tablet press with a tool for 13 mm biconvex tablets, the core coated with cellulose acetate was inserted centrally, the remaining 250 mg of jacket mixture were added and the jacket was pressed around the core.

### Example 22

### Multiparticulate Form

Emetic Pellets

| | |
|---|---|
| Emetine | 50 mg |
| Lactose | 15 mg |
| Microcrystalline cellulose PH 101 | 30 mg |
| Low-substituted hydroxypropylcellulose (LH31, Shin-Etsu) | 5 mg |

All the constituents were intimately mixed together for approx. 5 min in a suitable mixer. During mixing, the mixture was granulated with such a quantity of purified water that a moist, granulated mass was formed. The resultant granular product was extruded in a Nica extruder through a die with extrusion orifices of 1 mm, rounded for 5 min in a spheroniser, dried in a fluidized bed at 60° C. and classified by means of a 1.5 mm and a 0.5 mm screen.

Coating on Emetic Pellets

| | |
|---|---|
| Cellulose acetate with 39.8% acetate | 9.5 mg |
| Macrogol 3350 | 0.5 mg |
| Titanium dioxide | 0.5 mg |

Quantities stated per 100 mg of emetic pellets

Cellulose acetate and macrogol were dissolved to form a 3.8% solution in an acetone-water mixture (95:5 parts by weight), titanium dioxide was dispersed in the mixture and the cores were sprayed with the suspension in a fluidized bed unit until the mass of the coated pellets amounted to 110% of the weight of the introduced uncoated pellets.

Analgesic Pellets

| | |
|---|---|
| 0.5 mm nonpareils (sucrose-maize starch starter | 50 mg |
| pellets, supplied by Werner) | |
| Morphine sulfate pentahydrate | 60 mg |
| Povidone K30 | 30 mg |
| Capsaicin | 0.1 mg |
| Talcum | 9.9 mg |

Morphine sulfate and povidone were dissolved in purified water and talcum was dispersed in the solution. Capsaicin was dissolved as a 10% solution in ethanol and the solution was added to the suspension. The suspension was sprayed onto the nonpareils at 60° C. and dried. The pellets were classified by means of a 1.5 mm and a 0.5 mm screen.

Coating on Analgesic Pellets

| | |
|---|---|
| Ethylcellulose dispersion (Aquacoat ECD 30, | 10.0 mg |
| FMC Corporation) | |
| Glycerol monostearate | 2.0 mg |
| Talcum | 2.0 mg |
| Titanium dioxide | 1.0 mg |

Quantities stated per 150 mg of analgesic pellets, weight of ethylcellulose stated as the dry weight obtained from the 30% dispersion of the commercial product.

The ethylcellulose dispersion was mixed 1:0.5 with purified water and the glycerol monostearate was incorporated by stirring for at least two hours. Talcum and titanium dioxide were dispersed in 0.5 parts of water (calculated on the basis of the 1:0.5 mixture of the ethylcellulose dispersion) and mixed with the ethylcellulose dispersion. The analgesic pellets were sprayed with the dispersion in a fluidized bed unit until the mass of the coated pellets amounted to 110% of the weight of the introduced uncoated pellets.

Capsules

110 mg of coated emetic pellets and 165 mg of coated analgesic pellets per capsule were mixed and packaged in size 1 hard gelatine capsules.

### Example 23

### Multiparticulate Form

Antagonist Pellets

| | |
|---|---|
| Naloxone hydrochloride dihydrate | 20 mg |
| Lactose | 7 mg |
| Microcrystalline cellulose PH101 | 20 mg |
| Low-substituted hydroxypropylcellulose (LH31, Shin-Etsu) | 3 mg |

All the constituents were intimately mixed together for approx. 5 min in a suitable mixer. During mixing, the mixture was granulated with such a quantity of purified water that a moist, granulated mass was formed. The resultant granular product was extruded in a Nica extruder through a die with extrusion orifices of 1 mm, rounded for 5 min in a spheroniser, dried in a fluidized bed at 60° C. and classified by means of a 1.5 mm and a 0.5 mm screen.

Coating on Antagonist Pellets

| | |
|---|---|
| Cellulose acetate with 39.8% acetate | 9.5 mg |
| Macrogol 3350 | 0.5 mg |
| Titanium dioxide | 0.5 mg |

Quantities stated per 100 mg of emetic pellets

Cellulose acetate and macrogol were dissolved to form a 3.8% solution in an acetone-water mixture (95:5 parts by weight), titanium dioxide was dispersed in the mixture and the cores were sprayed with the suspension in a fluidized bed unit until the mass of the coated pellets amounted to 110% of the weight of the introduced uncoated pellets.

Analgesic Pellets

| | |
|---|---|
| 0.5 mm nonpareils (sucrose-maize starch starter | 50 mg |
| pellets, supplied by Werner) | |
| Morphine sulfate pentahydrate | 60 mg |
| Povidone K30 | 30 mg |
| Cayenne pepper | 5 mg |
| Talcum | 10 mg |

Morphine sulfate and povidone were dissolved in purified water and cayenne pepper and talcum were dispersed in the solution. The suspension was sprayed onto the nonpareils at 60° C. and dried. The pellets were classified by means of a 1.5 mm and a 0.5 mm screen.

Coating on Analgesic Pellets

| | |
|---|---|
| Ethylcellulose dispersion (Aquacoat ECD 30, | 10.0 mg |
| FMC Corporation) | |
| Glycerol monostearate | 2.0 mg |
| Talcum | 2.0 mg |
| Titanium dioxide | 1.0 mg |

Quantities stated per 150 mg of analgesic pellets, weight of ethylcellulose stated as the dry weight obtained from the 30% dispersion of the commercial product.

The ethylcellulose dispersion was mixed 1:0.5 with purified water and the glycerol monostearate was incorporated by stirring for at least two hours. Talcum and titanium dioxide were dispersed in 0.5 parts of water (calculated on the basis of the 1:0.5 mixture of the ethylcellulose dispersion) and mixed with the ethylcellulose dispersion. The analgesic pellets were sprayed with the dispersion in a fluidized bed unit until the mass of the coated pellets amounted to 110% of the weight of the introduced uncoated pellets.

Capsules

55 mg of coated antagonist pellets and 170 mg of coated analgesic pellets per capsule were mixed and packaged in size 2 hard gelatine capsules.

### Example 24

### Jacketed Tablets

Core

| | |
|---|---|
| Emetine hydrochloride pentahydrate | 60 mg |
| Hydrogenated castor oil (Cutina HR) | 40 mg |

Emetine hydrochloride pentahydrate and finely powdered hydrogenated castor oil were mixed and press-molded in a tablet press to form round, biconvex tables of a diameter of 6.5 mm.

Coating on Core

| | |
|---|---|
| Cellulose acetate with 39.8% acetate | 9.5 mg |
| Macrogol 3350 | 0.5 mg |

The coating constituents were dissolved as a 3.8% solution in an acetone-water mixture (95:5 parts by weight) and sprayed onto the cores.

Jacket

| | |
|---|---|
| Morphine sulfate pentahydrate | 60 mg |
| Methylhydroxypropylcellulose 100,000 mPa · s (Metolose | 100 mg |
| 90 SH 100,000, ShinEtsu) | |
| Microcrystalline cellulose (Avicel PH 102) | 165 mg |
| Lactose monohydrate | 164.9 mg |
| Capsaicin, micronised | 0.1 mg |
| Magnesium stearate | 5 mg |
| Colloidal silicon dioxide | 5 mg |

All the jacket constituents were mixed; approx. 250 mg of the mixture were placed in the tablet die in a tablet press with a tool for 13 mm biconvex tablets, the 6.5 mm core coated with cellulose acetate was inserted centrally, the remaining 250 mg of jacket mixture were added and the jacket was pressed around the core.

### Example 25

### Oral Osmotic Therapeutic System (OROS)

Active Ingredient Layer

| | |
|---|---|
| Morphine sulfate pentahydrate | 125 mg |
| Macrogol 200,000 | 280 mg |
| Povidone (MW_{N} 40,000) | 26 mg |
| Cayenne pepper | 15 mg |
| Magnesium stearate | 4 mg |

The morphine sulfate and macrogol were dry-mixed in a planetary mixer and then converted into a moist mass by slow addition of a solution of the povidone in 115 mg of ethanol and the mass was then pressed through a 0.8 mm screen. After 24 hours' drying at room temperature in a fume hood, the particles were pressed together with the magnesium stearate and cayenne pepper through a 1.0 mm screen and mixed in a container mixer.

Push Layer

| | |
|---|---|
| Methylhydroxypropylcellulose 6 mPa · s | 13 mg |
| Sodium chloride | 80 mg |
| Macrogol 7,000,000 | 166 mg |
| Magnesium stearate | 1 mg |

The sodium chloride, macrogol and half the methylhydroxypropylcellulose were dry-mixed for 3 minutes in a fluidized bed granulator and then granulated and dried by spraying on a solution of the second half of the methylhydroxypropylcellulose in 75 mg with introduction of hot air. The granular product was then pressed together with the magnesium stearate through a 2.5 mm screen in a Coil.

Emetic Layer

| | |
|---|---|
| Emetine | 50 mg |
| Hydrogenated castor oil (Cutina HR) | 50 mg |

Emetine and hydrogenated castor oil were precompressed in a tablet press with a 10 mm precompression punch to form approx. 250 mg compression moldings. The preliminary compression moldings were then comminuted by means of a crusher and a 1.0 mm screen.

Production of the 3 Layer Tablets

For each tablet, 100 mg of the granular product for the emetic layer, 260 mg of the push layer and 450 mg of the active ingredient layer were introduced in succession into the die of a suitable tablet press and press-molded to form a 3 layer tablet.

Coating on Core

| | |
|---|---|
| Cellulose acetate with 39.8% acetate | 38 mg |
| Macrogol 3350 | 2 mg |

The coating constituents were dissolved as a 3.8% solution in an acetone-water mixture (95:5 parts by weight) and sprayed onto the cores. Two 0.75 mm holes were drilled through the coating in order to connect the active ingredient layer with external environment of the system.

### Example 26

### Oral Osmotic Therapeutic System

The same procedure was used as in Example 25, except that the emetic layer was of the following composition:

| | |
|---|---|
| Emetine hydrochloride pentahydrate | 60 mg |
| Hydrogenated castor oil (Cutina HR) | 40 mg |

Emetine hydrochloride pentahydrate and hydrogenated castor oil were precompressed in a tablet press with a 10 mm precompression punch to form approx. 250 mg compression moldings. The preliminary compression moldings were then comminuted by means of a crusher and a 1.0 mm screen. All the other production steps proceeded as explained in Example 25.

### Example 27

### Jacketed Tablets

Core

| | |
|---|---|
| Naltrexone hydrochloride | 50 mg |
| Spray-dried lactose | 46 mg |
| Magnesium stearate | 2 mg |
| Colloidal silicon dioxide | 2 mg |

All the constituents were mixed and press-molded in a tablet press to form round, biconvex tablets of a diameter of 6.5 mm.

Coating on Core

| | |
|---|---|
| Cellulose acetate with 39.8% acetate | 9.5 mg |
| Macrogol 3350 | 0.5 mg |

The coating constituents were dissolved in an acetone-water mixture (95:5 parts by weight) and sprayed onto the cores.

Jacket

| | |
|---|---|
| Morphine sulfate pentahydrate | 60 mg |
| Methylhydroxypropylcellulose 100,000 mPa · s (Metolose 90 | 100 mg |
| SH 100,000, ShinEtsu) | |
| Xanthan, NF | 40 mg |
| Microcrystalline cellulose (Avicel PH 102) | 165 mg |
| Lactose monohydrate | 155 mg |
| Cayenne pepper | 10 mg |
| Magnesium stearate | 5 mg |
| Colloidal silicon dioxide | 5 mg |

All the jacket constituents were mixed; approx. 270 mg of the mixture were placed in the tablet die in a tablet press with a tool for 13 mm biconvex tablets, the core coated with cellulose acetate was inserted centrally, the remaining 270 mg of jacket mixture were added and the jacket was pressed around the core.

### Example 28

### Multiparticulate Form

Emetic Pellets

| | |
|---|---|
| Emetine | 50 mg |
| Lactose | 15 mg |
| Microcrystalline cellulose PH101 | 30 mg |
| Low-substituted hydroxypropylcellulose (LH31, Shin-Etsu) | 5 mg |

All the constituents were intimately mixed together for approx. 5 min in a suitable mixer. During mixing, the mixture was granulated with such a quantity of purified water that a moist, granulated mass was formed. The resultant granular product was extruded in a Nica extruder through a die with extrusion orifices of 1 mm, rounded for 5 min in a spheroniser, dried in a fluidized bed at 60° C. and classified by means of a 1.5 mm and a 0.5 mm screen.

Coating on Emetic Pellets

| | |
|---|---|
| Cellulose acetate with 39.8% acetate | 9.5 mg |
| Macrogol 3350 | 0.5 mg |
| Titanium dioxide | 0.5 mg |

Quantities stated per 100 mg of emetic pellets

Cellulose acetate and macrogol were dissolved to form a 3.8% solution in an acetone-water mixture (95:5 parts by weight), titanium dioxide was dispersed in the mixture and the cores were sprayed with the suspension in a fluidized bed unit until the mass of the coated pellets amounted to 110% of the weight of the introduced uncoated pellets.

Analgesic Pellets

| | |
|---|---|
| 0.5 mm nonpareils (sucrose-maize starch starter pellets, supplied | 50 mg |
| by Werner) | |
| Morphine sulfate pentahydrate | 60 mg |
| Povidone K30 | 30 mg |
| Capsaicin | 0.1 mg |
| Talcum | 9.9 mg |
| Xanthan | 30 mg |

Morphine sulfate and povidone were dissolved in purified water and half of the talcum was dispersed in the solution. Capsaicin was dissolved as a 10% solution in ethanol and the solution was added to the suspension. The suspension was sprayed at 55° C. in a Glatt Rotogranulator onto the rotating nonpareils, the xanthan was continuously introduced as a powder mixed with the second half of the talcum into the mass of moistened, rotating pellets. Once drying was complete, the pellets were classified using a 1.5 mm screen and a 0.5 mm screen.

Coating on Analgesic Pellets

| | |
|---|---|
| Ethylcellulose dispersion (Aquacoat ECD 30, FMC Corporation) | 12.0 mg |
| Glycerol monostearate | 2.4 mg |
| Talcum | 2.4 mg |
| Titanium dioxide | 1.2 mg |

Quantities stated per 180 mg of analgesic pellets, weight of ethylcellulose stated as the dry weight obtained from the 30% dispersion of the commercial product.

The ethylcellulose dispersion was mixed 1:0.5 with purified water and the glycerol monostearate was incorporated by stirring for at least two hours. Talcum and titanium dioxide were dispersed in 0.5 parts of water (calculated on the basis of the 1:0.5 mixture of the ethylcellulose dispersion) and mixed with the ethylcellulose dispersion. The analgesic pellets were sprayed with the dispersion in a fluidized bed unit until the mass of the coated pellets amounted to 110% of the weight of the introduced uncoated pellets.

Capsules

110 mg of coated emetic pellets and 198 mg of coated analgesic pellets per capsule were mixed and packaged in size 1 hard gelatine capsules.

### Example 29

### Multiparticulate Form

Emetic Pellets

| | |
|---|---|
| Emetine | 50 mg |
| Lactose | 15 mg |
| Microcrystalline cellulose PH101 | 30 mg |
| Low-substituted hydroxypropylcellulose (LH31, Shin-Etsu) | 5 mg |

All the constituents were intimately mixed together for approx. 5 min in a suitable mixer. During mixing, the mixture was granulated with such a quantity of purified water that a moist, granulated mass was formed. The resultant granular product was extruded in a Nica extruder through a die with extrusion orifices of 1 mm, rounded for 5 min in a spheroniser, dried in a fluidized bed at 60° C. and classified by means of a 1.5 mm and a 0.5 mm screen.

Coating on Emetic Pellets

| | |
|---|---|
| Cellulose acetate with 39.8% acetate | 9.5 mg |
| Macrogol 3350 | 0.5 mg |
| Titanium dioxide | 0.5 mg |

Quantities stated per 100 mg of emetic pellets

Cellulose acetate and macrogol were dissolved to form a 3.8% solution in an acetone-water mixture (95:5 parts by weight), titanium dioxide was dispersed in the mixture and the cores were sprayed with the suspension in a fluidized bed unit until the mass of the coated pellets amounted to 110% of the weight of the introduced uncoated pellets.

Antagonist Pellets

| | |
|---|---|
| Naloxone hydrochloride dihydrate | 20 mg |
| Lactose | 7 mg |
| Microcrystalline cellulose PH 101 | 20 mg |
| Low-substituted hydroxypropylcellulose (LH31, Shin-Etsu) | 3 mg |

All the constituents were intimately mixed together for approx. 5 min in a suitable mixer. During mixing, the mixture was granulated with such a quantity of purified water that a moist, granulated mass was formed. The resultant granular product was extruded in a Nica extruder through a die with extrusion orifices of 1 mm, rounded for 5 min in a spheroniser, dried in a fluidized bed at 60° C. and classified by means of a 1.5 mm and a 0.5 mm screen.

Coating on Antagonist Pellets

| | |
|---|---|
| Cellulose acetate with 39.8% acetate | 4.75 mg |
| Macrogol 3350 | 0.25 mg |
| Titanium dioxide | 0.25 mg |

Quantities stated per 50 mg of emetic pellets

Cellulose acetate and macrogol were dissolved to form a 3.8% solution in an acetone-water mixture (95:5 parts by weight), titanium dioxide was dispersed in the mixture and the cores were sprayed with the suspension in a fluidized bed unit until the mass of the coated pellets amounted to 110% of the weight of the introduced uncoated pellets.

Analgesic Pellets

| | |
|---|---|
| 0.5 mm nonpareils (sucrose-maize starch starter pellets, supplied | 50 mg |
| by Werner) | |
| Morphine sulfate pentahydrate | 60 mg |
| Povidone K30 | 30 mg |
| Capsaicin | 0.1 mg |
| Talcum | 9.9 mg |
| Xanthan | 30 mg |

Morphine sulfate and povidone were dissolved in purified water and half of the talcum was dispersed in the solution. Capsaicin was dissolved as a 10% solution in ethanol and the solution was added to the suspension. The suspension was sprayed at 55° C. in a Glatt Rotogranulator onto the rotating nonpareils, the xanthan was continuously introduced as a powder mixed with the second half of the talcum into the mass of moistened, rotating pellets. Once drying was complete, the pellets were classified by means of a 1.5 mm and a 0.5 mm screen.

Coating on Analgesic Pellets

| | |
|---|---|
| Ethylcellulose dispersion (Aquacoat ECD 30, FMC Corporation) | 12.0 mg |
| Glycerol monostearate | 2.4 mg |
| Talcum | 2.4 mg |
| Titanium dioxide | 1.2 mg |

Quantities stated per 180 mg of analgesic pellets, weight of ethylcellulose stated as the dry weight obtained from the 30% dispersion of the commercial product.

The ethylcellulose dispersion was mixed 1:0.5 with purified water and the glycerol monostearate was incorporated by stirring for at least two hours. Talcum and titanium dioxide were dispersed in 0.5 parts of water (calculated on the basis of the 1:0.5 mixture of the ethylcellulose dispersion) and mixed with the ethylcellulose dispersion. The analgesic pellets were sprayed with the dispersion in a fluidized bed unit until the mass of the coated pellets amounted to 110% of the weight of the introduced uncoated pellets.

Capsules

110 mg of coated emetic pellets, 55 mg of antagonist pellets and 198 mg of coated analgesic pellets with gel former per capsule were mixed and packaged in size 0 hard gelatine capsules.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. An abuse-safeguarded drug dosage form, comprising at least one active ingredient with potential for abuse, and at least two of the following components (a) through (d):
(a) at least one substance which irritates nasal passages and/or pharynx;
(b) at least one viscosity-increasing agent, which, together with a necessary minimum quantity of an aqueous liquid, forms a gel with an extract obtained from the dosage form, which gel remains visually distinguishable when introduced into a further quantity of an aqueous liquid;
(c) at least one antagonist for said at least one active ingredient with potential for abuse, and
(d) at least one emetic.

2. A dosage form according to claim 1, comprising at least three of said components (a) through (d).

3. A dosage form according to claim 2, comprising components (a), (b) and (c) or components (a), (b) and (d).

4. A dosage form according to claim 1, comprising all of components (a) through (d).

5. A dosage form according to claim 1, wherein said at least one active ingredient is selected from the group consisting of opiates, opioids, tranquillizers, stimulants and non-opiate narcotics.

6. A dosage form according to claim 5, wherein said at least one active ingredient is selected from the group consisting of N-{1-[2-(4-ethyl-5-oxo-2-tetrazolin-1-yl)ethyl]-4-methoxymethyl-4-piperidyl}propionanilide (alfentanil), 5,5-diallylbarbituric acid (allobarbital), allylprodine, alphaprodine, 8-chloro-1-methyl-6-phenyl-4H-[1,2,4]triazolo[4,3-a][1,4]-benzodiazepine (alprazolam), 2-diethylaminopropiophenone (amfepramone), (±)-α-methylphenethylamine (amphetamine), 2-(α-methylphenethylamino)-2-phenylacetonitrile (amphetaminil), 5-ethyl-5-isopentylbarbituric acid (amobarbital) anileridine, apocodeine, 5,5-diethylbarbituric acid (barbital), benzylmorphine, bezitramide, 7-bromo-5-(2-pyridyl)-1H-1,4-benzodiazepine-2(3H)-one (bromazepam), 2-bromo-4-(2-chlorophenyl)-9-methyl-6H-thieno[3,2-f][1,2,4]triazolo[4,3-a][1,4]diazepine (brotizolam), 17-cyclopropylmethyl-4,5α-epoxy-7α[(S)-1-hydroxy-1,2,2-trimethyl-propyl]-6-methoxy-6,14-endo-ethanomorphinan-3-ol (buprenorphine), 5-butyl-5-ethylbarbituric acid (butobarbital), butorphanol, (7-chloro-1,3-dihydro-1-methyl-2-oxo-5-phenyl-2H-1,4-benzodiazepin-3-yl) dimethylcarbamate (camazepam), (1S,2S)-2-amino-1-phenyl-1-propanol (cathine/D-norpseudoephedrine), 7-chloro-N-methyl-5-phenyl-3H-1,4-benzodiazepin-2-ylamine 4-oxide (chlordiazepoxide), 7-chloro-1-methyl-5-phenyl-1H-1,5-benzodiazepine-2,4(3H,5H)-dione (clobazam), 5-(2-chlorophenyl)-7-nitro-1H-1,4-benzodiazepin-2(3H)-one (clonazepam), clonitazene, 7-chloro-2,3-dihydro-2-oxo-5-phenyl-1H-1,4-benzodiazepine-3-carboxylic acid (clorazepate), 5-(2-chlorophenyl)-7-ethyl-1-methyl-1H-thieno[2,3-e][1,4]diazepin-2(3H)-one (clotiazepam), 10-chloro-11b-(2-chlorophenyl)-2,3,7,11b-tetrahydrooxazolo[3,2-d][1,4]benzodiazepin-6(5H)-one (cloxazolam), (−)-methyl-[3β-benzoyloxy-2β(1αH,5αH)-tropane carboxylate] (cocaine), 4,5α-epoxy-3-methoxy-17-methyl-7-morphinen-6α-ol (codeine), 5-(1-cyclohexenyl)-5-ethylbarbituric acid (cyclobarbital) cyclorphan, cyprenorphine, 7-chloro-5-(2-chlorophenyl)-1H-1,4-benzodiazepin-2(3H)-one (delorazepam), desomorphine, dextromoramide, (+)-(1-benzyl-3-dimethylamino-2-methyl-1-phenylpropyl)propionate (dextropropoxyphene), dezocine, diampromide, diamorphone, 7-chloro-1-methyl-5-phenyl-1H-1,4-benzodiazepin-2(3H)-one (diazepam), 4,5α-epoxy-3-methoxy-17-methyl-6α-morphinanol (dihydrocodeine), 4,5α-epoxy-17-methyl-3,6a-morphinandiol (dihydromorphine), dimenoxadol, dimephetamol, dimethylthiambutene, dioxaphetyl butyrate, dipipanone, (6aR,10aR)-6,6,9-trimethyl-3-pentyl-6a,7,8,10a-tetrahydro-6H-benzo[c]chromen-1-ol (dronabinol), eptazocine, 8-chloro-6-phenyl-4H-[1,2,4]triazolo[4,3-a][1,4]benzodiazepine (estazolam), ethoheptazine, ethylmethylthiambutene, ethyl [7-chloro-5-(2-fluorophenyl)-2,3-dihydro-2-oxo-1H-1,4-benzodiazepine-3-carboxylate] (ethyl loflazepate), 4,5α-epoxy-3-ethoxy-17-methyl-7-morphinen-6α-ol (ethylmorphine), etonitazene, 4,5α-epoxy-7α-(1-hydroxy-1-methylbutyl)-6-methoxy-17-methyl-6,14-endo-etheno-morphinan-3-ol (etorphine), N-ethyl-3-phenyl-8,9,10-trinorbornan-2-ylamine (fencamfamine), 7-[2-(α-methylphenethylamino)ethyl]-theophylline) (fenethylline), 3-(α-methylphenethylamino)propionitrile (fenproporex), N-(1-phenethyl-4-piperidyl)propionanilide (fentanyl), 7-chloro-5-(2-fluorophenyl)-1-methyl-1H-1,4-benzodiazepin-2(3H)-one (fludiazepam), 5-(2-fluorophenyl)-1-methyl-7-nitro-1H-1,4-benzodiazepin-2(3H)-one (flunitrazepam), 7-chloro-1-(2-diethylaminoethyl)-5-(2-fluorophenyl)-1H-1,4-benzodiazepin-2(3H)-one (flurazepam), 7-chloro-5-phenyl-1-(2,2,2-trifluoroethyl)-1H-1,4-benzodiazepin-2(3H)-one (halazepam), 10-bromo-11b-(2-fluorophenyl)-2,3,7,11b-tetrahydro[1,3]oxazolo[3,2-d][1,4]benzodiazepin-6(5H)-one (haloxazolam), heroin, 4,5α-epoxy-3-methoxy-17-methyl-6-morphinanone (hydrocodone), 4,5α-epoxy-3-hydroxy-17-methyl-6-morphinanone (hydromorphone), hydroxypethidine, isomethadone, hydroxymethylmorphinan, 11-chloro-8,12b-dihydro-2,8-dimethyl-12b-phenyl-4H-[1,3]oxazino[3,2-d][1,4]benzodiazepine-4,7(6H)-dione (ketazolam), 1-[4-(3-hydroxyphenyl)-1-methyl-4-piperidyl]-1-propanone (ketobemidone), (3S,6S)-6-dimethylamino-4,4-diphenylheptan-3-yl acetate (levacetylmethadol (LAAM)), (−)-6-dimethylamino-4,4-diphenol-3-heptanone (levomethadone), (−)-17-methyl-3-morphinanol (levorphanol), levophenacylmorphane, lofentanil, 6-(2-chlorophenyl)-2-(4-methyl-1-piperazinylmethylene)-8-nitro-2H-imidazo[1,2-a][1,4]-benzodiazepin-1(4H)-one (loprazolam), 7-chloro-5-(2-chlorophenyl)-3-hydroxy-1H-1,4-benzodiazepin-2(3H)-one (lorazepam), 7-chloro-5-(2-chlorophenyl)-3-hydroxy-1-methyl-1H-1,4-benzodiazepin-2(3H)-one (lormetazepam), 5-(4-chlorophenyl)-2,5-dihydro-3H-imidazo[2,1-a]isoindol-5-ol (mazindol), 7-chloro-2,3-dihydro-1-methyl-5-phenyl-1H-1,4-benzodiazepine (medazepam), N-(3-chloropropyl)-α-methylphenethylamine (mefenorex), meperidine, 2-methyl-2-propyltrimethylene dicarbamate (meprobamate), meptazinol, metazocine, methylmorphine, N,α-dimethylphenethylamine (metamphetamine), (±)-6-dimethylamino-4,4-diphenol-3-heptanone (methadone), 2-methyl-3-o-tolyl-4(3H)-quinazolinone (methaqualone), methyl [2-phenyl-2-(2-piperidyl)acetate] (methylphenidate), 5-ethyl-1-methyl-5-phenylbarbituric acid (methylphenobarbital), 3,3-diethyl-5-methyl-2,4-piperidinedione (methyprylon), metopon, 8-chloro-6-(2-fluorophenyl)-1-methyl-4H-imidazo[1,5-a][1,4]benzodiazepine (midazolam), 2-(benzhydrylsulfinyl)acetamide (modafinil), 4,5α-epoxy-17-methyl-7-morphinen-3,6α-diol (morphine), myrophine, (±)-trans-3-(1,1-dimethylheptyl)-7,8,10,10α-tetrahydro-1-hydroxy-6,6-dimethyl-6H-dibenzo-[b,d]pyran-9(6αH)-one (nabilone), nalbuphene, nalorphine, narceine, nicomorphine, 1-methyl-7-nitro-5-phenyl-1H-1,4-benzodiazepin-2(3H)-one (nimetazepam), 7-nitro-5-phenyl-1H-1,4-benzodiazepin-2(3H)-one (nitrazepam), 7-chloro-5-phenyl-1H-1,4-benzodiazepin-2(3H)-one (nordazepam), norlevorphanol, 6-dimethylamino-4,4-diphenyl-3-hexanone (normethadone), normorphine, norpipanone, the exudation from plants belonging to the species Papaver somniferum (opium), 7-chloro-3-hydroxy-5-phenyl-1H-1,4-benzodiazepin-2(3H)-one (oxazepam), (cis-trans)-10-chloro-2,3,7,11b-tetrahydro-2-methyl-11b-phenyloxazolo[3,2-d][1,4]benzodiazepin-6-(5H)-one (oxazolam), 4,5α-epoxy-14-hydroxy-3-methoxy-17-methyl-6-morphinanone (oxycodone), oxymorphone, plants and parts of plants belonging to the species Papaver somniferum (including the subspecies setigerum) (Papaver somniferum), papaveretum, 2-imino-5-phenyl-4-oxazolidinone (pernoline), 1,2,3,4,5,6-hexahydro-6,11-dimethyl-3-(3-methyl-2-butenyl)-2,6-methano-3-benzazocin-8-ol (pentazocine), 5-ethyl-5-(1-methylbutyl)-barbituric acid (pentobarbital), ethyl (1-methyl-4-phenyl-4-piperidinecarboxylate) (pethidine), phenadoxone, phenomorphane, phenazocine, phenoperidine, piminodine, pholcodeine, 3-methyl-2-phenylmorpholine (phenmetrazine), 5-ethyl-5-phenylbarbituric acid (phenobarbital), α,α-dimethylphenethylamine (phentermine), 7-chloro-5-phenyl-1-(2-propynyl)-1H-1,4-benzodiazepin-2(3H)-one (pinazepam), α-(2-piperidyl)benzhydryl alcohol (pipradrol), 1′-(3-cyano-3,3-diphenylpropyl)[1,4′-bipiperidine]-4′-carboxamide (piritramide), 7-chloro-1-(cyclopropylmethyl)-5-phenyl-1H-1,4-benzodiazepin-2(3H)-one (prazepam), profadol, proheptazine, promedol, properidine, propoxyphene, N-(1-methyl-2-piperidinoethyl)-N-(2-pyridyl)propionamide, methyl {3-[4-methoxycarbonyl-4-(N-phenylpropanamido)piperidino]propanoate} (remifentanil), 5-sec-butyl-5-ethylbarbituric acid (secbutabarbital), 5-allyl-5-(1-methylbutyl)-barbituric acid (secobarbital), N-{4-methoxymethyl-1-[2-(2-thienyl)ethyl]-4-piperidyl}propionanilide (sufentanil), 7-chloro-2-hydroxy-methyl-5-phenyl-1H-1,4-benzodiazepin-2(3H)-one (temazepam), 7-chloro-5-(1-cyclohexenyl)-1-methyl-1H-1,4-benzodiazepin-2(3H)-one (tetrazepam), ethyl (2-dimethylamino-1-phenyl-3-cyclohexene-1-carboxylate) (tilidine, cis and trans)), tramadol, 8-chloro-6-(2-chlorophenyl)-1-methyl-4H-[1,2,4]triazolo[4,3-a][1,4]benzodiazepine (triazolam), 5-(1-methylbutyl)-5-vinylbarbituric acid (vinylbital), (1R*,2R*)-3-(3-dimethylamino-1-ethyl-2-methyl-propyl)-phenol, (1R,2R,4S)-2-(dimethylamino)methyl-4-(p-fluorobenzyloxy)-1-(m-methoxyphenyl)cyclohexanol, esters and ethers of any of the foregoing, and pharmaceutically acceptable salts and solvates of any of the foregoing.

7. A dosage form according to claim 5, which comprises an antagonist for said at least one active ingredient with potential for abuse.

8. A dosage form according to claim 7, wherein said at least one active ingredient is selected from the group consisting of N-{1-[2-(4-ethyl-5-oxo-2-tetrazolin-1-yl)ethyl]-4-methoxymethyl-4-piperidyl}propionanilide (alfentanil), allylprodine, alphaprodine, 2-diethylaminopropiophenone (amfepramone), (±))-α-methylphenethylamine (amphetamine), 2-(α-methylphenethylamino)-2-phenylacetonitrile (amphetaminil), anileridine, apocodeine, benzylmorphine, bezitramide, 17-cyclopropylmethyl-4,5α-epoxy-7α[(S)-1-hydroxy-1,2,2-trimethyl-propyl]-6-methoxy-6,14-endo-ethanomorphinan-3-ol (buprenorphine), butorphanol, (1S,2S)-2-amino-1-phenyl-1-propanol (cathine/D-norpseudoephedrine), clonitazene, (−)-methyl-[3β-benzoyloxy-2β(1αH,5αH)-tropane carboxylate] (cocaine), 4,5α-epoxy-3-methoxy-17-methyl-7-morphinen-6α-ol (codeine), cyclorphan, cyprenorphine, desomorphine, dextromoramide, (+)-(1-benzyl-3-dimethylamino-2-methyl-1-phenylpropyl) propionate (dextropropoxyphene), dezocine, diampromide, diamorphone, 4,5α-epoxy-3-methoxy-17-methyl-6α-morphinanol (dihydrocodeine), 4,5α-epoxy-17-methyl-3,6a-morphinandiol (dihydromorphine), dimenoxadol, dimephetamol, dimethylthiambutene, dioxaphetyl butyrate, dipipanone, (6aR,10aR)-6,6,9-trimethyl-3-pentyl-6a,7,8,10a-tetrahydro-6H-benzo[c]chromen-1-ol (dronabinol), eptazocine, ethoheptazine, ethylmethylthiambutene, 4,5α-epoxy-3-ethoxy-17-methyl-7-morphinen-6α-ol (ethylmorphine), etonitazene, 4,5α-epoxy-7α-(1-hydroxy-1-methylbutyl)-6-methoxy-17-methyl-6,14-endo-etheno-morphinan-3-ol (etorphine), N-ethyl-3-phenyl-8,9,10-trinorbornan-2-ylamine (fencamfamine), 7-[2-(α-methylphenethylamino)ethyl]-theophylline) (fenethylline), 3-(α-methylphenethylamino)propionitrile (fenproporex), N-(1-phenethyl-4-piperidyl)propionanilide (fentanyl), heroin, 4,5α-epoxy-3-methoxy-17-methyl-6-morphinanone (hydrocodone), 4,5α-epoxy-3-hydroxy-17-methyl-6-morphinanone (hydromorphone), hydroxypethidine, isomethadone, hydroxymethylmorphinan, 1-[4-(3-hydroxyphenyl)-1-methyl-4-piperidyl]-1-propanone (ketobemidone), (3S,6S)-6-dimethylamino-4,4-diphenylheptan-3-yl acetate (levacetylmethadol (LAAM)), (−)-6-dimethylamino-4,4-diphenol-3-heptanone (levomethadone), (−)-17-methyl-3-morphinanol (levorphanol), levophenacylmorphane, lofentanil, 5-(4-chlorophenyl)-2,5-dihydro-3H-imidazo[2,1-a]isoindol-5-ol (mazindol), N-(3-chloropropyl)-α-methylphenethylamine (mefenorex), meperidine, meptazinol, metazocine, methylmorphine, N,α-dimethylphenethylamine (metamphetamine), (±)-6-dimethylamino-4,4-diphenol-3-heptanone (methadone), methyl [2-phenyl-2-(2-piperidyl)acetate] (methylphenidate), 3,3-diethyl-5-methyl-2,4-piperidinedione (methyprylon), 2-(benzhydrylsulfinyl)acetamide (modafinil), 4,5α-epoxy-17-methyl-7-morphinen-3,6α-diol (morphine), myrophine, (±)-trans-3-(1,1-dimethylheptyl)-7,8,10,10α-tetrahydro-1-hydroxy-6,6-dimethyl-6H-dibenzo-b,d]pyran-9(6αH)-one (nabilone), nalbuphene, narceine, nicomorphine, norlevorphanol, 6-dimethylamino-4,4-diphenyl-3-hexanone (normethadone), normorphine, norpipanone, the exudation from plants belonging to the species Papaver somniferum (opium), 4,5α-epoxy-14-hydroxy-3-methoxy-17-methyl-6-morphinanone (oxycodone), oxymorphone, plants and parts of plants belonging to the species Papaver somniferum (including the subspecies setigerum) (Papaver somniferum), papaveretum, 2-imino-5-phenyl-4-oxazolidinone (pernoline), 1,2,3,4,5,6-hexahydro-6,11-dimethyl-3-(3-methyl-2-butenyl)-2,6-methano-3-benzazocin-8-ol (pentazocine), ethyl (1-methyl-4-phenyl-4-piperidinecarboxylate) (pethidine), phenadoxone, phenomorphane, phenazocine, phenoperidine, piminodine, pholcodeine, 3-methyl-2-phenylmorpholine (phenmetrazine), α,α-dimethylphenethylamine (phentermine), α-(2-piperidyl)benzhydryl alcohol (pipradrol), 1′-(3-cyano-3,3-diphenylpropyl)[1,4′-bipiperidine]-4′-carboxamide (piritramide), profadol, proheptazine, promedol, properidine, propoxyphene, N-(1-methyl-2-piperidinoethyl)-N-(2-pyridyl)propionamide, methyl {3-[4-methoxycarbonyl-4-(N-phenylpropanamido)piperidino]propanoate} (remifentanil), N-{4-methoxymethyl-1-[2-(2-thienyl)ethyl]-4-piperidyl}propionanilide (sufentanil), ethyl (2-dimethylamino-1-phenyl-3-cyclohexene-1-carboxylate) (tilidine, cis and trans)), tramadol, (1R*,2R*)-3-(3-dimethylamino-1-ethyl-2-methyl-propyl)-phenol, (1R,2R,4S)-2-(dimethylamino)methyl-4-(p-fluorobenzyloxy)-1-(m-methoxyphenyl)cyclohexanol, esters and ethers of any of the foregoing, and physiologically acceptable salts and solvates of any of the foregoing.

9. A dosage form according to claim 5, which comprises at least one stimulant selected from the group consisting of amphetamine, norpseudoephedrine, methylphenidate, and physiologically acceptable salts and solvates thereof.

10. A dosage form according to claim 1, comprising a component (a) irritant substance which causes at least one sensation selected from the group consisting of burning, itching, an urge to sneeze, and an increased formation of secretions.

11. A dosage form according to claim 1, comprising a component (a) irritant comprising at least one constituent of at least one hot substance drug selected from the group consisting of Allii sativi bulbus, Asari rhizoma cum herba, Calami rhizoma, Capsici fructus, Capsici fructus acer, Curcumae longae rhizoma, Curcumae xanthorrhizae rhizoma, Galangae rhizoma, Myristicae semen, Piperis nigri fructus, Sinapis albae semen, Sinapis nigri semen, Zedoariae rhizome, and Zingiberis rhizome.

12. A dosage form according to claim 11, wherein said at least one hot substance drug is selected from the group consisting of Capsici fructus, Capsici fructus acer, and Piperis nigri fructus.

13. A dosage form according to claim 11, wherein said at least one constituent is an o-methoxy(methyl)phenol compound, an acid amide compound, a mustard oil, or a sulfide compound.

14. A dosage form according to claim 11, wherein said at least one constituent is selected from the group consisting of myristicin, elemicin, isoeugenol, β-asarone, safrole, gingerols, xanthorrhizol, capsaicinoids, piperine, and glucosinolates.

15. A dosage form according to claim 14, wherein said at least one constituent is capsaicin, trans-piperine, or a non-volatile mustard oil selected from the group consisting of p-hydroxybenzyl mustard oil, methylmercapto mustard oil and methylsulfonyl mustard oil.

16. A dosage form according to claim 1, comprising at least one component (b) viscosity increasing agent selected from the group consisting of microcrystalline cellulose with 11 wt. % carboxymethylcellulose sodium, carboxymethylcellulose sodium, polyacrylic acid, locust bean flour, citrus pectin, waxy maize starch, sodium alginate, guar flour, iota-carrageenan, karaya gum, gellan gum, galactomannan, tara stone flour, propylene glycol alginate, apple pectin, lemon peel pectin, sodium hyaluronate, tragacanth, tara gum, fermented polysaccharide welan gum, and xanthan gum.

17. A dosage form according to claim 1, comprising ≧5 mg of at least one component (b) viscosity-increasing agent per dosage form.

18. A dosage form according to claim 1, comprising a component (c) opiate or opioid antagonist selected from the group consisting of naloxone, naltrexone, nalmefene, nalide, nalmexone, nalorphine, naluphine, and physiologically acceptable salts and solvates of any of the foregoing.

19. A dosage form according to claim 18, wherein the component (c) antagonist comprises a neuroleptic selected from the group consisting of haloperidol, promethazine, fluphenazine, perphenazine, levomepromazine, thioridazine, perazine, chlorpromazine, chlorprothixine, zuclopentixol, flupentixol, prothipendyl, zotepine, benperidol, pipamperone, melperone and bromperidol.

20. A dosage form according to claim 1, comprising a component (d) emetic comprising apomorphine or at least one constituent of ipecacuanha root.

21. A dosage form according to claim 20, wherein said at least one constituent of ipecacuanha root is emetine.

22. A dosage form according to claim 1, wherein said at least one active ingredient is spatially separate from at least one component selected from the group consisting of components (c) and components (d); said at least one active ingredient being present in a subunit (A) and said at least one component selected from the group consisting of components (c) and components (d) being present in a subunit (B), and wherein, when the dosage form is correctly administered to an individual, said at least one component selected from the group consisting of components (c) and components (d) is not released from said subunit (B) in an effective amount in the body of said individual.

23. A dosage form according to claim 22, the subunits (A) and (B) both have the same multiparticulate form selected from the group consisting of microtablets, microcapsules, micropellets, granules, spheroids, beads, and pellets, with said subunits (A) and (B) having the same size and shape.

24. A dosage form according to claim 23, wherein the multiparticulate subunits (A) and (B) are press-molded together to form tablets, or packaged together in capsules, or suspended together in a liquid or a gel.

25. A dosage form according to claim 23, wherein the multiparticulate subunits (A) and (B) are visually indistinguishable from one another.

26. A dosage form according to claim 22, wherein the subunits (A) and (B) are arranged in layers relative to one another.

27. A dosage form according to claim 26, wherein the layered subunits (A) and (B) are arranged vertically or horizontally relative to one another.

28. A dosage form according to claim 22, wherein subunit (B) forms a core which is completely enclosed by subunit (A).

29. A dosage form according to claim 22, wherein subunit (A) forms a core which is enclosed by subunit (B), and subunit (B) comprises at least one channel which leads from the core to the surface of the dosage form.

30. A dosage form according to claim 26, further comprising at least one optionally swellable separation layer arranged between the layers of subunits (A) and (B).

31. A dosage form according to claim 26, having the form of a tablet.

32. A dosage form according to claim 26, wherein the free surface of subunit (B) is entirely coated with at least one barrier layer which prevents release of said at least one component selected from the group consisting of (c) and (d).

33. A dosage form according to claim 32, wherein at least part of subunit (A) is also coated with said barrier layer.

34. A dosage form according to claim 26, further comprising a push layer between subunits (A) and (B), and wherein all free surfaces of subunits (A) and (B) and the push layer are covered by a semi-permeable coating which is permeable to a release medium but substantially impermeable to the at least one active ingredient and said at least one component selected from the group consisting of (c) and (d), and wherein said semi-permeable coating is provided in the area of subunit (A) with at least one opening for release of the at least one active ingredient.

35. A dosage form according to claim 23, wherein subunit (A) has the form of a tablet with a peripheral edge face and two main faces, and wherein said peripheral edge face and optionally one of the two main faces is covered with a barrier layer containing an emetic.

36. A dosage form according to claim 1, wherein at least part of said at least one active ingredient is present in a delayed-release form.

37. A dosage form according to claim 1, having an orally administrable form.

38. A dosage form according to claim 37, further comprising at least one coating resistant to gastric juices.

38. A dosage form according to claim 23, comprising component (a) and/or (b) in at least one subunit A and/or at least one subunit B.
